(19) United States
(12) Patent Application Publication
(10) Pub. No.: US 2005/0026215 A1
(43) Pub. Date: Feb. 3, 2005

(54) Method for the prediction of an epitope

(76) Inventors: Paul F. Predki, Hamden, CT (US); Hong Guo, Guilford, CT (US)

Correspondence Address: JONES DAY, 222 EAST 41ST ST, NEW YORK, NY, 10017, US

(21) Appl. No.: 10/622,675

(22) Filed: Jul. 17, 2003

(51) Int. Cl. G01N 33/53; G06F 19/00; G01N 33/48; G01N 33/50

(52) U.S. Cl. 435/7.1; 702/19

(57) ABSTRACT

The invention provides methods for the prediction of an epitope in a target protein. An epitope of the target protein can be bound by a given molecule, e.g., by an antibody. In particular, the methods of the invention comprise (i) identifying a plurality of cross-reactive proteins, i.e., proteins that can be bound by the same molecule, e.g., by the same antibody, as the target protein using, e.g., protein microarrays; and (ii) comparing the amino acid sequences of the target protein and the cross-reactive proteins with each other to identify windows of sequence homology, wherein the windows of sequence homology correspond to the epitope.

### 1. FIELD OF THE INVENTION

The invention relates to a method for the prediction of a binding site of a molecule in a target protein. In particular, the invention relates to a bioinformatics algorithm for the prediction of a binding site of a molecule in a target protein using sequence information of the target protein and other cross-reactive proteins that are bound by the same molecule. Specifically, the invention relates to the prediction of an epitope in a target protein.

### 2. BACKGROUND OF THE INVENTION

The greater use of antibodies as therapeutics, as well as the burgeoning field of proteomics and its demand for high-throughput protein analysis, have been accompanied by an increasing demand for large numbers of antibodies with high and well characterized specificities. An array containing every protein for the relevant organism represents the ideal format for an assay to test antibody specificity, since it allows the simultaneous screening of thousands of proteins in relatively normalized quantities.

Although approximately 10,000 antibodies are available from commercial sources, there are still tens of thousands of proteins for which antibodies are not available (Kesnezow and Hoheisel, 2002, Biotechniques Suppl, 14-23). Furthermore, new applications such as antibody arrays (Schweitzer et al., 2002, Nat Biotechnol 20, 359-65; Haab et al., 2001, Genome Biol 2; Knezevic et al., 2001, Proteomics 1, 1271-8; Moody et al., 2001, Biotechniques 31, 186-90, 192-4) and antibody therapeutics (Huston & George, 2001, Hum Antibodies 10, 127-42; Pastan & Kreitman, 2002, Curr Opin Investig Drugs 3, 1089-91) have increased the demand for more specific antibodies in order to reduce cross-reactivity and side effects. Conventional strategies for generating antibodies by animal immunization are unlikely to meet these demands, although recombinant antibody technologies such as phage (McCafferty et al., 1990, Nature 348, 552-4; Marks et al., 1991, J Mol Biol 222, 581-97; Griffiths et al., 1994, EMBO J 13, 3245-60), ribosome (Hanes & Pluckthun, 1997, Proc Natl Acad Sci USA 94, 4937-42) and mRNA (Roberts & Szostak, 1997, Proc Natl Acad Sci USA 94, 12297-302) display have demonstrated the potential to relieve this bottleneck. New methodologies such as affibodies (Nord et al., 2000, J Biotechnol 80, 45-54) and aptamers (Hesselberth et al., 2000, J Biotechnol 74, 15-25) have also been added to the repertoire of strategies for high-throughput generation of new affinity reagents. The advent of these new technologies has the potential to shift the rate limiting step in antibody development from antibody generation to antibody specificity screening.

An ideal format for determining antibody specificity would be one in which an antibody is simultaneously screened against all proteins that could possibly cross-react with the cognate antigen. Snyder and coworkers recently described the preparation of a functional protein microarray that closely approaches this ideal Zhu et al. (2001, Science 293, 2101-5). More than 80% of the 6,280 annotated (Harrison et al., 2002, Nucleic Acid Res 30, 1083-1090) genes from the yeast Saccharomyces cerevisae genome were cloned, overexpressed, purified and arrayed in an addressable format on glass slides Zhu et al. (2001, Science 293, 2101-5). This work represented the first time that the majority of proteins in a proteome had been individually isolated and transferred simultaneously to a solid surface. This “whole-proteome” microarray has proven to be a powerful tool for high-throughput and comprehensive measurements of protein-protein, protein-lipid, and protein-small molecule interactions (Zhu et al., 2001, Science 293, 2101-5; Zhu et al., 2000, Nat Genet 26, 283-9; and Zhu & Snyder, 2001, Curr Opin Chem Biol 5, 40-5). This technology will also be a powerful means of comprehensive profiling of antibody specificity.

The present invention provides new methods for the prediction of an epitope in a target protein based on amino acid sequence comparisons of the target protein with the amino acid sequences of cross-reactive proteins that are bound by the same antibody as the target molecule.

Citation or identification of any reference in this application shall not be considered as admission that such reference is available as prior art to the present invention.

### 3. SUMMARY OF THE INVENTION

The present invention provides methods for the identification of a region in a target protein that can be specifically bound by a particular molecule. In specific embodiments, the invention provides methods for the prediction of an epitope in a target protein that can be bound by a particular antibody.

The invention provides a method for predicting a binding site or part of a binding site in a target protein, wherein said binding site can be bound by a molecule, and wherein the method comprises the following steps: (a) comparing, for each of a plurality of cross-reactive proteins, each of a first plurality of amino acid sequences in a region of said target protein with each of a second plurality of amino acid sequences in a region of said cross-reactive protein, wherein each said cross-reactive protein can be bound by said molecule; and (b) identifying an amino acid sequence in said first plurality of amino acid sequences that exhibits the highest average sequence homology score, said average score being based upon the sequence homologies to an amino acid sequence in each of said second plurality of amino acid sequences in regions of said cross-reactive proteins, wherein said identified amino acid sequence in said first plurality of amino acid sequences is predicted to be said binding site or said part of a binding site in said target protein. In certain embodiments, the first plurality of amino acid sequences comprises successive overlapping amino acid sequences spanning said region of said target protein. In certain embodiments, the said plurality of amino acid sequences of each said cross-reactive protein comprises successive overlapping amino acid sequences spanning said region of said cross-reactive protein. In certain, more specific, embodiments, said successive overlapping amino acid sequence span said region of said target protein at an amino acid interval of 1 amino acid. In certain, more specific, embodiments, said successive overlapping amino acid sequences span said region of said cross-reacting protein at a amino acid interval of 1 amino acid.

In certain embodiments, the invention provides a method for predicting at least part of a binding site of a molecule in a target protein, said method comprising: (a) evaluating the degree of homology between each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein of the plurality of cross-reactive proteins can be bound by the molecule, and (ii) n is between 6 and 25; (b) performing step (a) for each cross-reactive protein of the plurality of cross-reactive proteins; (c) identifying, for each n-amino acid window in the target protein, the highest degree of sequence homology with an n-amino acid window in a cross-reactive protein for each cross-reactive protein; (d) identifying the n-amino acid window(s) in the target protein that have the highest average of the highest degrees of sequence homologies identified in step (c), wherein said identified n-amino acid window(s) comprises at least part of the binding site(s) in the target protein.

In even other embodiments, the invention provides a method for predicting a binding site or part of a binding site of a molecule in a target protein, said method comprising: (a) comparing each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein of the plurality of cross-reactive proteins can be bound by the molecule, and (ii) n is between 6 and 25; (b) assigning a score for each n-amino acid window comparison of step (a), wherein the score reflects the degree of sequence homology between the two n-amino acid windows compared; (c) performing steps (a) and (b) for each cross-reactive protein of the plurality of cross-reactive proteins; (d) identifying the highest scores assigned in step (b) of each n-amino acid window in the target protein for each cross-reactive protein; and (e) identifying the n-amino acid window(s) in the target protein that have the highest average score(s), wherein said identified n-amino acid window(s) comprises at least part of the binding site(s) in the target protein.

In certain, more specific, embodiments, the binding site is an epitope and the molecule is an antibody.

In certain, more specific, embodiments, the degree of sequence homology in the methods of the invention reflects the degree of sequence identity.

In certain, more specific, embodiments, the degree of sequence homology reflects the degree of sequence similarity.

In certain embodiments, the plurality of n-amino acid windows in the target protein comprises successive, overlapping amino acid sequences spanning a region of the target protein. In certain, more specific embodiments, said successive overlapping amino acid sequence span said region of said target protein at an amino acid interval of 1 amino acid.

In certain embodiments, the plurality of n-amino acid windows in each cross-reactive protein comprises successive overlapping amino acid sequences spanning a region of the cross-reactive protein. In certain, more specific embodiments, said successive overlapping amino acid sequence span said region of said cross-reactive protein at an amino acid interval of 1 amino acid.

In certain embodiments, the region of the target protein has been identified as containing the binding site. In certain embodiments, the region of the cross-reactive protein has been identified as containing the binding site. In certain, more specific, embodiments, the region of the target protein consists of the entire contiguous amino acid sequence of the target protein. In certain, more specific, embodiments, the region of the cross-reactive protein consists of the entire contiguous amino acid sequence of the cross-reactive protein. In certain embodiments, the region of the target protein has been identified as being on the surface of the folded target protein. In certain embodiments, the region of the cross-reactive protein has been identified as being on the surface of the folded cross-reactive protein.

In certain embodiments, the method of the invention is computer-implemented.

The invention also provides a computer system comprising a processor, and a memory coupled to said processor and encoding one or more programs, wherein said one or more programs cause the processor to carry out the method of the invention.

The invention further provides a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of the invention.

### BRIEF DESCRIPTION OF FIGURES

FIG. 1) Fluorescent images of antibody probings of the yeast proteome microarray. Subarrays for anti-Cdc11, anti-Nap1, anti-Sed3 show the antibodies reacting with their cognate proteins, which are boxed (thick lines) in white. For anti-Myc, a typical subarray was chosen to show the lack of signal obtained with this antibody. The rectangular box (thinner lines) is drawn around the spots representing the dilution series (0.1-12.8 pg/spot) of pure GST that is printed on each array for quantitation purposes. White spots indicate greater signal intensity, darker spots indicate weaker signal intensity.

FIG. 2) Analysis of anti-Hda1 binding to the yeast proteome microarray. A) Portions of microarray images showing the spots corresponding to the Hda1 protein and the 7 proteins that gave significant signals with the anti-Hda1 antibody. Images on the right are from an array that was probed with the goat polyclonal antibody against Hda1 and then with a fluorescently labeled anti-goat antibody. Images on the left are from an array that was probed with only the fluorescently labeled anti-goat antibody as control (Ctrl). B) Western analysis of proteins detected with the anti-Hda1 antibody on the proteome arrays. The Western blot was probed with anti-Hda1 antibody. C) Peptide inhibition of anti-Hda1 binding on yeast proteome microarrays. Images on the left are from an array that was probed with the anti-Hda1 antibody alone. Images in the middle are from an array that was probed in the presence of the Hda1 immunizing peptide. Images on the right are from an array that was probed in the presence of a peptide with an unrelated sequence.

FIG. 3A) Maximal average sequence identity of cross-reactive proteins for antibodies targeted against Tpk1p. 3B) Maximal average sequence identity of cross-reactive proteins for antibodies targeted against CDC11p. 3C) Maximal average sequence identity of cross-reactive proteins for antibodies targeted against Rud3p. x-axis=sequence window number, y-axis=maximal average identity. Methods as described in text. Regions of maximal similarity are indicated by arrows. 3D) Sequence alignment of and around the immunogenic peptide region with best matches from each of the cross reacting peptides. The immunogenic peptide is underlined in the Hda1 sequence. The 8 amino acid window with highest maximal sequence identity between all cross-reacting proteins is depicted in bold.

FIG. 4A) Analysis of anti-Pep12 binding to the yeast proteome microarray. Portions of microarray images showing the spots corresponding to the Pep12 protein and the 3 proteins that gave significant signals with the anti-Pep12 antibody. B) Western blot probed with anti-Pep12 antibody. C) Western blot probed with anti-GST. Positions of MW standards, GST-fusion of Yor036W, and endogenous Yor036W^{wt} are shown.

FIG. 5) Western analysis of anti-Clb5 cross-reactivity. GST (lane 1) and GST-Yfl045C (lane 2) were electrophoresed and blotted as described in Experimental Protocols. Blots were probed with A) anti-GST and B) anti-Clb5 antibodies. Positions of MW standards, GST, and GST-Yfl045C are shown

FIG. 6) Fluorescent image from the anti-Nap1 antibody probing of the yeast proteome microarray. Subarray 24 of the array is shown as a typical example of the binding of this antibody to the proteins on the array.

FIG. 7) Western analysis of anti-Nap1 cross-reactivity. Blots were probed with A) anti-GST and B) anti-Nap1 antibodies. Positions of MW standards, GST-Ykr048C-Nap1, GST-Ybl082C, GST-Ypr183W, and Yhr111W are shown.

FIG. 8) Samples are pure GST (lane 1), GST-Yjl164C-Tpk1 (lane 2, 76 kDa), GST-Ykl166C (lane 3, 76 kDa), GST-Ypl203W (lane 4, 74 kDa), and GST-Yil033C (lane 5, 77 kDa). (Tpk1wt predicted MW=43.6 kDa).

FIG. 9) Microarray-based protein-protein interaction showing the interaction between Tpk1 and Yil033C and interaction between Yil033C with Ypl203W and with Ykl166C on the array.

FIG. 10) Western of GST-Yjr076C-Cdc11 (lane 1), GST-Yml048W (lane 2), GST-Ylr301W (lane 3), GST-Yor042W (lane 4), GST-Yll039C (lane 5) and GST-Yor144C (lane 6). A) Western blot probed with anti-GST antibody. B) Western blot probed with anti-Cdc11. Positions of molecular weight standards shown.

FIG. 11) Samples are pure GST (lane 1), GST-Yjl164C-Tpk1 (lane 2), GST-Ykl166C (lane 3), GST-Yfr014C (lane 4), GST-Ynr023C (lane 5), GST-Ypl203W (lane 6), GST-Ylr173W (lane 7), GST-Yol019W (lane 8), GST-Yel016C (lane 9) and GST-Yil033C (lane 10).

FIG. 12) Maximal average sequence identity of cross-reactive proteins for antibodies targeted against HDA1. x-axis=sequence window number, y-axis=maximal average identity. Methods as described in text. Regions of maximal similarity are indicated by arrows.

FIG. 13) Diagram illustrating an exemplary embodiment of a computer system useful for implementing the methods of this invention.

### 4. DEFINITIONS, CONVENTIONS AND ABBREVIATIONS

As used herein, the term “binding site” refers to a region of a protein to which a molecule binds. Different binding sites in proteins for molecules can be of different sizes. A binding site can be bound by a molecule, such as, but not limited to, an antibody, a protein, a polypeptide, a peptide, a nucleic acid, a small organic molecule, an inorganic molecule, a lipid, or a sugar. A molecule can bind to a binding site with different affinities, such as, but not limited to, with a binding affinity of at least 1 M⁻¹, 10 M⁻¹, 10² M⁻¹, 10³ M⁻¹, 5×10³ M⁻¹, 10⁴ M⁻¹, 5×10⁴ M⁻¹, 10⁵ M⁻¹, 5×10⁵ M⁻¹, 10⁶ M⁻¹, 5×10⁶ M⁻¹, 10⁷ M⁻¹, 5×10⁷ M⁻¹, 10⁸ M⁻¹, 5×10⁸ M⁻¹, 10⁹ M⁻¹, 5×10⁹ M⁻¹, 10¹⁰ M⁻¹, 5×10¹⁰ M⁻¹, 10¹¹ M⁻¹, 5×10¹¹ M⁻¹, 10¹² M⁻¹, 5×10¹² M⁻¹, 10¹³ M⁻¹, 5×10¹³ M⁻¹, 10¹⁴ M⁻¹, or at least 10¹⁵ M⁻¹. In a preferred embodiment, a molecule binds to a binding site with an affinity between 10³ M⁻¹ to 10¹² M⁻¹.

As used herein, the term “epitope” refers to a region of a protein to which an antibody binds. Different epitopes can be of different sizes.

As used herein, the term “region of a protein” refers to a portion of the protein that is contiguous in space. In specific embodiments, a region of a protein consists of a plurality of amino acids that are contiguous in sequence.

As used herein, the phrase “an n-amino acid window corresponds to a binding site in a protein” is used to describe that the amino acid sequence of the n-amino acid window encompasses the entire binding site or part of the binding site in the protein.

Abbreviation

GST Glutathione S-transferase
GPTS 3-glycidooxypropyltrimethoxysilane
n-amino acid window Designates an amino acid window of a protein, which contains n amino acids
m_{target} Designates the amino acid position of the first amino acid of an n-amino acid window in a target protein
m_{cross-reactive} Designates the amino acid position of the first amino acid of an n-amino acid window in a cross-reactive protein

### 5. DETAILED DESCRIPTION OF THE INVENTION

The invention relates to methods for the identification of one or more binding sites in a target protein that can be bound by a particular molecule. In certain, more specific embodiments, the invention provides methods for the prediction of an epitope in a target protein that can be bound by a particular antibody.

In certain embodiments, the invention provides a method for predicting a binding site in a target protein, wherein said binding site can be bound by a molecule, said method comprising the following steps: (a) comparing, for each of a plurality of cross-reactive proteins, each of a first plurality of amino acid sequences in a region of said target protein with each of a second plurality of amino acid sequences in a region of said cross-reactive protein, wherein each said cross-reactive protein can be bound by said molecule; and (b) identifying an amino acid sequence in said first plurality of amino acid sequences that exhibits the highest average sequence homology score, said average score being based upon the sequence homologies to an amino acid sequence in each of said second plurality of amino acid sequences in regions of said cross-reactive proteins, wherein said identified amino acid sequence in said first plurality of amino acid sequences is predicted to be said binding site in said target protein.

In certain embodiments, a method for predicting at least part of a binding site of a molecule in a target protein comprises the following steps: (a) evaluating the degree of homology between each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein of the plurality of cross-reactive proteins can be bound by the molecule, and (ii) n is between 6 and 25; (b) performing step (a) for each cross-reactive protein of the plurality of cross-reactive proteins; (c) identifying, for each n-amino acid window in the target protein, the highest degree of sequence homology with an n-amino acid window in a cross-reactive protein for each cross-reactive protein; and (d) identifying the n-amino acid window(s) in the target protein that have the highest average of the highest degrees of sequence homologies identified in step (c), wherein said identified n-amino acid window(s) comprises at least part of the binding site(s) in the target protein.

In certain embodiments, a method of the invention comprises the following steps: (a) comparing each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein of the plurality of cross-reactive proteins can be bound by the molecule, and (ii) n is between 6 and 25; (b) assigning a score for each n-amino acid window comparison of step (a), wherein the score reflects the degree of sequence homology between the two sequences compared; (c) performing steps (a) and (b) for each cross-reactive protein of the plurality of cross-reactive proteins; (d) identifying the highest scores assigned in step (b) of each n-amino acid window in the target protein for each cross-reactive protein; and (e) identifying the n-amino acid window(s) in the target protein that have the highest average score(s), wherein said identified n-amino acid window(s) comprises at least part of the binding site(s) in the target protein.

In certain embodiments n is at least 5, 10, 15, 20, 25, 30, 35, 40, 45, 50, 60, 70, 80, 90, or at least 100. In certain embodiments n is at most 5, 10, 15, 20, 25, 30, 35, 40, 45, 50, 60, 70, 80, 90, or at least 100. In certain embodiments, n is 5, 6, 7, 8, 9, 10, 11, 12, 13, 14, 15, 16, 17, 18, 19, 20, 21, 22, 23, 24, 25, 26, 27, 28, 29, or 30. In a preferred embodiment, n is between 6 and 25.

In certain, more specific embodiments, a method of the invention further includes identifying the plurality of cross-reactive proteins. The plurality of cross-reactive proteins can be identified by any method known to the skilled artisan. For illustrative methods for identifying the plurality of cross-reactive proteins, see section 5.3. In a specific embodiment, the cross-reactive proteins are identified using a protein microarray. In certain, more specific embodiments, the molecule is an antibody and the binding site is an epitope.

In a specific embodiment, the size of the window is 8, i.e., n of the n-amino acid window equals 8.

In certain embodiments, a method of the invention comprises the identification of proteins that can be specifically bound by the same molecule as the target protein, i.e., cross-reactive proteins. Since the target protein and the different cross-reactive proteins are bound by the same molecule, the target protein and the cross-reactive proteins each have at least one binding site that can be bound by the molecule. Without being bound by theory, the binding sites of the target protein and the cross-reactive proteins that can be bound by the same molecule consist of 6 to 25 contiguous amino acids, wherein the amino acid sequences of the binding site in the target protein and of each of the cross-reactive protein are sufficiently homologous, similar or identical to each other to support specific binding by the same molecule.

In certain embodiments, the degree of homology of amino acid sequences can be evaluated by determining the degree of amino acid identity, e.g., the percentage of amino acid identity, between the sequences in a sequence comparison. In certain embodiments, a sequence comparison can be performed by an alignment of the two sequences with each other with or without the introduction of gaps (see section 5.1.2) to determine the degree of sequence identity. In other embodiments, the degree of homology of amino acid sequences can be evaluated by determining the sequence similarity between the amino acid sequences. In certain embodiments, sequence similarity between amino acid sequences in a sequence comparison can be evaluated using any amino acid substitution matrix known to the skilled artisan. Based on the amino acid substitution matrix, values are assigned to each amino acid substitution between the sequences. In a specific embodiment, higher values are assigned if structural and/or functional properties of the amino acids' side-chains are similar or identical to each other. Highest values are assigned if the amino acids are identical. Structural and/or functional properties of the amino acids' side-chains are similar between the amino acids, e.g., if an aromatic amino acid is substituted for another aromatic amino acid, if an acidic amino acid is substituted for another acidic amino acid, is a basic amino acid is substituted for another basic amino acid, and if an aliphatic amino acid is substituted for another aliphatic amino acid. In specific embodiments, an amino acid substitution matrix that can be used with the methods of the invention is the PAM matrix (see, e.g., Dayhoff, Schwartz and Orcutt, 1978, A model of evolutionary change in proteins. Matrices for detecting distant relationships. In Atlas of protein sequence and structure, (Dayhoff, M. O., ed.), vol. 5, pp. 345-358. National biomedical research foundation Washington D.C.). The degree of homology can be expressed as a score. Exemplary methods for determining a score for a sequence comparison are set forth in section 5.1.1.

### 5.1 PREDICTION OF EPITOPES BOUND BY AN ANTIBODY

In certain embodiments, the invention relates to methods for the identification of one or more epitopes in a target protein that can be bound by a particular antibody. In certain, more specific, embodiments, a method of the invention also comprises the step of identifying a plurality of cross-reactive proteins that can be bound by the same antibody as the target protein. Since the target protein and the different cross-reactive proteins can be specifically recognized and bound by the same antibody, the target protein and the cross-reactive proteins each have at least one epitope that can be bound by the antibody. The structures of the epitopes of the target protein and the cross-reactive proteins that can be bound by the antibody have to be sufficiently similar to each other to support binding by the same antibody. As the structure of an epitope in a protein is determined by the primary structure of its amino acid sequence, the amino acid sequences of the epitope in the target protein and the amino acid sequences of the epitopes of each of the cross-reactive protein are sufficiently homologous to each other. Sequence homology can be evaluated by determining sequence identity or sequence similarity. In certain embodiments, the amino acid sequences of the epitope in the target protein and of each of the cross-reactive protein are at least 60%, at least 65%, at least 70%, at least 75%, at least 80%, at least 85%, at least 90%, at least 95%, at least 98% or at least 99% identical to each other. In other embodiments, the amino acid sequences of the epitope in the target protein and of each of the cross-reactive protein are sufficiently similar to each other, e.g., at least 60%, at least 65%, at least 70%, at least 75%, at least 80%, at least 85%, at least 90%, at least 95%, at least 98% or at least 99% similar. Without being bound by theory, the epitopes of the target protein and the cross-reactive proteins that can be bound by the same antibody consist of 6 to 25 contiguous amino acids.

In certain embodiments, the antibody binds to the epitope in the target protein with an affinity ofat least 1 M⁻¹, 10 M⁻¹, 10² M⁻¹, 10³ M⁻¹, 10⁴ M⁻¹, 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, 10¹⁴ M⁻¹, or at least 10¹⁵ M⁻¹. In certain embodiments, the antibody binds to the epitope in the cross-reactive protein with an affinity of at least 1 M⁻¹, 10 M⁻¹, 10² M⁻¹, 10³ M⁻¹, 10⁴ M⁻¹, 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, 10¹⁴ M⁻¹, or at least 10¹⁵ M⁻¹.

In certain embodiments, the antibodv is a monoclonal antibody or an antigen-binding fragment thereof. In other embodiments, the methods of the invention are performed to identify epitopes that are bound by polyclonal antibodies. In certain embodiments, the antibody is a humanized antibody. In certain embodiments, the antibody can be, but is not limited to, a chimeric antibody, a single chain antibody, or a Fab fragment.

The cross-reactive proteins that can be bound by the same antibody as the target protein can be identified by any method known to the skilled artisan. In certain embodiments, cross-reactive proteins are identified by screening a plurality of proteins on protein microarrays with the antibody. Illustrative methods for identifying cross-reactive proteins are described in section 5.3. In certain embodiments, cross-reactive proteins are identified using immunological methods such as, but not limited to, immunoprecipitation, Western blot analysis, and affinity chromatography.

In certain embodiments, the invention provides a method for predicting an epitope of a target protein that can be bound by an antibody, wherein the method comprises the following steps: (a) comparing, for each of a plurality of cross-reactive proteins, each of a first plurality of amino acid sequences in a region of said target protein with each of a second plurality of amino acid sequences in a region of said cross-reactive protein, wherein each said cross-reactive protein can be bound by said antibody; and (b) identifying an amino acid sequence in said first plurality of amino acid sequences that exhibits the highest average sequence homology score, said average score being based upon the sequence homologies to an amino acid sequence in each of said pluralities of amino acid sequences in regions of said cross-reactive proteins, wherein said identified amino acid sequence in said first plurality of amino acid sequences is predicted to be said epitope in said target protein.

In certain embodiments, a method for predicting at least part of an epitope of a target protein that can be bound by an antibody comprises the following steps: (a) evaluating the degree of homology between each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein of the plurality of cross-reactive proteins can be bound by the antibody, and (ii) n is between 6 and 25; (b) performing step (a) for each cross-reactive protein of the plurality of cross-reactive proteins; (c) identifying, for each n-amino acid window in the target protein, the highest degree of sequence homology with an n-amino acid window in a cross-reactive protein for each cross-reactive protein; and (d) identifying the n-amino acid window(s) in the target protein that have the highest average of the highest degrees of sequence homologies identified in step (c), wherein said identified n-amino acid window(s) comprises at least part of the epitope in the target protein.

In certain embodiments, a method for predicting at least part of an epitope of a target protein that can be bound by an antibody comprises the following steps: (a) comparing each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein of the plurality of cross-reactive proteins can be bound by the antibody, and (ii) n is between 6 and 25; (b) assigning a score for each n-amino acid window comparison of step (a), wherein the score reflects the degree of sequence homology between the two sequences compared; (c) performing steps (a) and (b) for each cross-reactive protein of the plurality of cross-reactive proteins; (d) identifying the highest scores of each n-amino acid window in the target protein for each cross-reactive protein; and (e) identifying the n-amino acid window(s) in the target protein that have the highest average score(s), wherein said identified n-amino acid window(s) comprises at least part of the epitope in the target protein.

In certain embodiments, the plurality of n-amino acid windows in the target protein contains n-amino acid windows of a region of the target protein, wherein the region of the target protein is known to encompass the epitope. In certain embodiments, the region of the target protein is contiguous in space and may contain one or more contiguous amino acid sequences. In an illustrative embodiment, two amino acid sequences of the target protein form together in space a region of the protein that contains the epitope, wherein the two amino acid sequences are not adjacent to each other in sequence but the two amino acid sequences are adjacent to each other in space. In other embodiments, the region of the target protein is contiguous in sequence.

In certain embodiments, the plurality of n-amino acid windows in the cross-reactive protein contains n-amino acid windows of a region of the cross-reactive protein, wherein the region of the cross-reactive protein is known to encompass the epitope. In certain embodiments, the region of the protein is contiguous in space and may contain one or more contiguous amino acid sequences. In an illustrative embodiment, two amino acid sequences of the cross-reactive protein form together in space a region of the protein that contains the epitope, wherein the two amino acid sequences are not adjacent to each other in sequence but the two amino acid sequences are adjacent to each other in space. In other embodiments, the region of the cross-reactive protein is contiguous in sequence.

In certain embodiments, the plurality of n-amino acid windows in the target protein comprises successive overlapping amino acid sequences spanning a region of the target protein wherein the region is contiguous in sequence. In certain embodiments, the interval between successive overlapping amino acid sequences is 1, 2, 3, 4, 5, 6, 7, 8, 9, 10, 11, 12, 13, 14, 15, 16, 17, 18, 19, 20, 21, 22, 23, 24, or 25 amino acids. The interval between two successive overlapping n-amino acid windows is the number of amino acids between the first amino acids of two successive n-amino acid windows plus 1. In certain embodiments, the region of the target protein has been identified as containing the epitope, has been identified as being on the surface of the target protein, or has been identified as being more antigenic than the remainder of the protein. In certain embodiments, the plurality of n-amino acid windows in a cross-reactive protein comprises successive overlapping amino acid sequences spanning a region of the cross-reactive protein. In certain embodiments, the interval between successive overlapping amino acid sequences in a cross-reactive protein is 1, 2, 3, 4, 5, 6, 7, 8, 9, 10, 11, 12, 13, 14, 15, 16, 17, 18, 19, 20, 21, 22, 23, 24, or 25 amino acids. The interval between two successive overlapping n-amino acid windows is the number of amino acids between the first amino acids of two successive n-amino acid windows plus 1. In certain embodiments, the region of a cross-reactive protein has been identified as containing the epitope, has been identified as being on the surface of the cross-reactive protein, or has been identified as being more antigenic than the remainder of the cross-reactive protein.

In certain, more specific embodiments, a method of the invention further comprises plotting the average of the maximum scores as a function of the position of the n-amino acid window in the target protein to identify the n-amino acid window(s) in the target protein with the highest average of the highest scores identified in step (d). In certain embodiments, the score reflects the sequence identity between the two n-amino acid windows compared. In other embodiments, the score reflects the sequence similarity between the two n-amino acid windows compared. In certain embodiments, the plurality of n-amino acid windows of the target protein contains all n-amino acid windows of the target protein. In certain embodiments, the plurality of n-amino acid windows of the cross-reactive proteins contains all n-amino acid windows of the cross-reactive protein.

In certain embodiments, the n-amino acid windows of the target protein to be compared with the n-amino acid windows of the cross-reactive proteins are located in a particular region of the target protein. In certain embodiments, the n-amino acid windows of a cross-reactive protein to be compared with n-amino acid windows of the target protein are located in a particular region of the cross-reactive protein. The particular region can be contiguous in space or contiguous in sequence. If the particular region is contiguous in space but not contiguous in sequence, the plurality of n-amino acid windows consists n-amino acid windows from two or more amino acid sequences of the target protein.

In certain embodiments, a subset of n-amino acid windows of the target protein and/or the cross-reactive protein is from a region of the protein that has been determined to be antigenic or hydrophilic. In certain embodiments, a subset of n-amino acid windows of the target protein and/or the cross-reactive protein is from a region of the protein that has been determined to be on the surface of the protein. In certain embodiments, a subset of n-amino acid windows of the target protein and/or the cross-reactive protein is from a region of the protein that has been determined to encompass the epitope.

In a specific embodiment, if the region of the target protein that contains the epitope has already been determined, only n-amino acid windows within the antigenic region are scanned and compared against the cross-reactive proteins. Regions in a protein that contain epitopes can be determined by any method known to the skilled artisan, and any such method can be combined with the methods of the invention. Exemplary methods for identifying a region in a protein that harbors an epitope include the following. Deletion mutants of the protein of interest can be tested for binding by the antibody. If the antibody fails to bind to a particular deletion mutant, the deletion affects the epitope. In a specific embodiment, if the antibody fails to bind to a deletion mutant of the target protein, the deleted region of the target protein in the mutant form of the target protein harbors the epitope. In certain other embodiments, fragments of the protein can be tested for binding by the antibody. The fragment that is bound by the antibody harbors the epitope. Different strategies can be employed to predict whether an amino acid sequence of a protein is on the surface of the protein and is thus more likely to contain the epitope. Such strategies include, but are not limited to, x-ray crystallography, Circular Dichroism (CD) spectra, and hydrophilicity plots.

In a specific embodiment, if the region of the cross-reactive protein that encompasses the epitope has already been determined, only n-amino acid windows within the antigenic region are compared with the n-amino acid windows of the target protein. Regions in a protein that contain epitopes can be determined by any method known to the skilled artisan, and any such method can be combined with the methods of the invention. Exemplary methods for identifying a region in a protein that harbors an epitope include the following. Deletion mutants of the cross-reactive protein of interest can be tested for binding by the antibody. If the antibody fails to bind to a particular deletion mutant, the deletion affects the epitope. In a specific embodiment, if the antibody fails to bind to a deletion mutant of the cross-reactive protein, the deleted region of the cross-reactive protein in the mutant form of the cross-reactive protein harbors the epitope. In certain other embodiments, fragments of the cross-reactive protein can be tested for binding by the antibody. The fragment that is bound by the antibody harbors the epitope. Different strategies can be employed to predict whether an amino acid sequence of a cross-reactive protein is on the surface of the protein and is thus more likely to contain the epitope. Such strategies include, but are not limited to, x-ray crystallography, Circular Dichroism (CD) spectra, and hydrophilicity plots.

In certain embodiments, a subset of the n-amino acid windows of the target protein is compared to each n-amino acid window of each cross-reactive protein. In other embodiments, a subset of the n-amino acid windows of the target protein is compared to a subset of the n-amino acid windows of each cross-reactive protein. In even other embodiments, a subset of the n-amino acid windows of the target protein is compared to a subset of the n-amino acid windows of some of the cross-reactive protein and to each n-amino acid window of the other cross-reactive proteins of the plurality of cross-reactive proteins.

In certain embodiments, each of the n-amino acid windows of the target protein is compared to each n-amino acid window of each cross-reactive protein. In other embodiments, each of the n-amino acid windows of the target protein is compared to a subset of the n-amino acid windows of each cross-reactive protein. In even other embodiments, each of the n-amino acid windows of the target protein is compared to a subset of the n-amino acid windows of some of the cross-reactive protein and to all n-amino acid windows of the other cross-reactive proteins of the plurality of cross-reactive proteins.

In certain embodiments, a subset of n-amino acid windows of the target protein and/or the cross-reactive protein represents at least 5%, 10%, 15%, 20%, 25%, 30%, 35%, 40%, 45%, 50%, 55%, 60%, 65%, 70%, 75%, 80%, 85%, 90%, 95%, 98% of a target protein or a cross-reactive protein, respectively. In certain embodiments, a subset of n-amino acid windows of the target protein and/or the cross-reactive protein represents at most 5%, 10%, 15%, 20%, 25%, 30%, 35%, 40%, 45%, 50%, 55%, 60%, 65%, 70%, 75%, 80%, 85%, 90%, 95%, 98% of a target protein or a cross-reactive protein, respectively.

All successively overlapping n-amino acid windows of the target protein or a region thereof can be scanned and compared against each of a plurality of n-amino acid windows of the cross-reactive proteins or a region thereof by any method known to the skilled artisan. In certain embodiments, all successively overlapping n-amino acid windows of a cross-reactive protein or a region thereof can be scanned and compared against each of a plurality of n-amino acid windows of the target protein-reactive proteins or a region thereof by any method known to the skilled artisan.

In certain embodiments, all successively overlapping n-amino acid windows of the target protein or a region thereof can be scanned and compared against each of a plurality of successively overlapping n-amino acid windows of the cross-reactive proteins or a region thereof by any method known to the skilled artisan. In certain embodiments, the n-amino acid window is constituted by 6, 7, 8, 9, 10, 11, 12, 13, 14, 15, 16, 17, 18, 19, 20, 21, 22, 23, 24, or 25 contiguous amino acids, i.e., n equals 6, 7, 8, 9, 10, 11, 12, 13, 14, 15, 16, 17, 18, 19, 20, 21, 22, 23, 24, or 25.

In a specific embodiment, the first n-amino acid window of the target protein (m_{target}=1; the first amino acid of the n-amino acid window is at amino acid position 1 of the target protein) is compared to the first n-amino acid window of the first cross-reactive protein (m_{cross-reactive}=1; the first amino acid of the n-amino acid window is at amino acid position 1 of the cross-reactive protein); the first n-amino acid window of the target protein is compared to the second n-amino acid of the first cross-reactive protein (m_{cross-reactive}=2); the first n-amino acid window of the target protein is compared to the third n-amino acid of the first cross-reactive protein (m_{cross-reactive}=3); the first n-amino acid window of the target protein is compared to all other n-amino acid of the first cross-reactive protein to and including the n-amino acid window starting at m_{cross-reactive}={(number of amino acids in the cross-reactive protein) minus n}. In the same manner, the second n-amino acid window of the target protein (m_{target}=2) and all other n-amino acid windows of the target protein to and including the n-amino acid window of the target protein starting at m_{target}={(number of amino acids in the target protein) minus n} are each compared to all n-amino acid windows of the first cross-reactive protein. In the same manner, all n-amino acid windows of the target protein are scanned and compared against all n-amino acid windows of the other cross-reactive proteins. In this embodiment, the interval between the n-amino acid windows in the target protein as well as the interval between n-amino acid windows in the cross-reactive protein is 1.

In certain embodiments, for each comparison of two n-amino acid windows a score is assigned. The score can depend on the degree of sequence identity (e.g., the percentage of identical amino acids between the sequences compared), or sequence similarity between the two sequences of the two n-amino acid windows. Different scoring algorithms can be used with the methods of the invention. Exemplary scoring algorithms are described in section 5.1.1. In addition to primary sequence information, any other information may figure into the score obtained for a particular n-amino acid window.

In certain embodiments, the degree of homology is evaluated for each comparison of two n-amino acid windows. In certain embodiments, the degree of homology is evaluated by determining the degree of sequence identity (e.g., the percentage of identical amino acids between the sequences compared), or sequence similarity between the two sequences of the two n-amino acid windows being compared.

For each n-amino acid window of the target protein the highest score for the first cross-reactive protein is determined; for each n-amino acid window of the target protein the highest score for the second cross-reactive protein is determined; for each n-amino acid window of the target protein the highest score for the third cross-reactive protein is determined; for each n-amino acid window of the target protein the highest score for all other cross-reactive proteins determined. Thus, each n-amino acid window in the target protein has assigned to it as many highest scores as there are cross-reactive proteins.

Subsequently, the average of the highest scores is determined for each n-amino acid window of the target protein. In certain embodiments, each highest score is weighted equally in calculating the average highest score. In other embodiments, in calculating the average of the highest scores, the highest score of each cross-reactive protein is weighted dependent on the affinity of the antibody to the cross-reactive protein. Thus, the higher the affinity of the antibody for a particular cross-reactive protein, the more weight has the highest score of that cross-reactive protein in calculating the average of the highest scores. The affinity of an antibody to a protein can be determined by any method known to the skilled artisan. For exemplary methods, see section 5.8

In certain embodiments, the average of the highest scores is plotted as a function of the position of the n-amino acid window in the target protein, i.e., m. The peaks in the plot identify regions of the protein that are candidates for epitopes that are bound by the antibody. In a specific embodiment, the peak that represents the maximum average of the highest scores identifies the epitope that is bound by the antibody. In certain embodiments, other methods are used in combination with the methods of the invention to identify the epitope in the target protein that is bound by the antibody. Any method known to the skilled artisan for the prediction of antigenic sites in a protein can be used to obtain additional information to decide which of the peaks corresponds to the epitope. In certain embodiments, Western blot analysis of fragments or deletion mutants using the antibody are performed to identify the larger region of the protein that harbors the epitope. In other embodiments, one or more of the following factors can optionally be considered in determining whether a peak in the plot corresponds to the epitope. Such factors include, but are not limited to, the antigenic index of the n-amino acid window as determined by the method of Hopp and Woods (1981, Proc. Natl. Acad. Sci. USA 86:152-156), the method of Kolaskar and Tongaonkar, (1990, FEBS Letters 276:172-174; see, e.g., homepage of EMBOSS (The European Molecular Biology Open Software Suite)), and the probability that the n-amino acid window that corresponds to the peak is on the surface of the folded protein as determined by a hydrophilicity plot.

In certain embodiments, more than one binding site or part of a binding site is identified using the methods of the invention. If two or more n-amino acid windows are identified as having each the highest average of the highest degrees of homologies then these n-amino acid windows are all identified as binding sites or parts of binding sites that can be bound by the molecule. In certain embodiments, the degree of homology is the same if any difference between the degrees of homologies is at most 10%, at most 5%, at most 1%, at most 0.5%, at most 0.1%, at most 0.05% or at most 0.01% of the value of the degree of homology. In a specific embodiment, if the degree of homology is expressed as percentage identity, the degree of homology is the same if the difference between the two degrees of homology is at most 10%, at most 5%, at most 1%, at most 0.5%, at most 0.1%, at most 0.05% or at most 0.01% sequence identity.

In certain embodiments, more than one binding site or part of a binding site is identified using the methods of the invention. If two or more n-amino acid windows are identified as having each the highest average of the highest scores then these n-amino acid windows are identified as binding sites or parts of binding sites that can be bound by the molecule. In certain embodiments, the score is the same if any difference between the scores is at most 10%, at most 5%, at most 1%, at most 0.5%, at most 0.1%, at most 0.05% or at most 0.01% of the score.

In a specific embodiment, if the antibody binds to the target protein only under denaturing conditions, the probability that the n-amino acid window that corresponds to the peak is on the surface of the folded protein is not factored into the decision which peak corresponds to the epitope. Under non-denaturing conditions, hydrophobic regions of the folded protein are often buried inside the protein and are not accessible to an antibody. Under denaturing conditions, however, these regions may be on the surface of the denatured protein and are thus accessible to an antibody.

Depending on the computer system used, the individual comparisons of n-amino acid windows can be processed concurrently or subsequently.

### 5.1.1 Scoring Algorithm

In certain embodiments, the degree of homology is evaluated for each comparison of two n-amino acid windows. In certain embodiments, the degree of homology is evaluated by determining the degree of sequence identity (e.g., the percentage of identical amino acids between the sequences compared), or sequence similarity between the two sequences of the two n-amino acid windows being compared.

In certain embodiments, for each comparison of two n-amino acid windows a score is assigned. The score depends, e.g., on the degree of amino acid sequence identity (e.g., the percentage of identical amino acids between the sequences being compared) or amino acid sequence similarity between the two sequences of the two n-amino acid windows.

In certain embodiments, the score is a function of the degree of amino acid identity, e.g., the percentage of amino acid identity, between the sequences in a sequence comparison. In certain embodiments, a sequence comparison can be performed by an alignment of the two sequences with each other with or without the introduction of gaps (see section 5.1.2). In other embodiments, the score is a function of the sequence similarity between the amino acid sequences (e.g., the n-amino acid windows being compared). Sequence similarity between amino acid sequences in a sequence comparison can be evaluated using any amino acid substitution matrix known to the skilled artisan. In certain embodiments, based on an amino acid substitution matrix, values are assigned to each amino acid substitution between the sequences. In a specific embodiment, higher values are assigned if structural and/or functional properties of the amino acids' side-chains are similar or identical to each other. Highest values are assigned if the amino acids are identical. Structural and/or functional properties of the amino acids' side-chains are similar between the amino acids, e.g. if an aromatic amino acid is substituted for another aromatic amino acid, if an acidic amino acid is substituted for another acidic amino acid, is a basic amino acid is substituted for another basic amino acid, and if an aliphatic amino acid is substituted for another aliphatic amino acid. In specific embodiments, an amino acid substitution matrix that can be used with the methods of the invention is the PAM matrix (see, e.g., Dayhoff, Schwartz and Orcutt, 1978, A model of evolutionary change in proteins. Matrices for detecting distant relationships. In Atlas of protein sequence and structure, (Dayhoff, M. O., ed.), vol. 5, pp. 345-358. National biomedical research foundation Washington D.C.). The degree of homology can be expressed as a score. Exemplary methods for determining a score for a sequence comparison are set forth herein.

Different scoring algorithms can be used with the methods of the invention to determine the score. In a specific embodiment, the score reflects the degree of amino acid sequence identity between the amino acid sequences of two n-amino acid windows compared (see section 5.1.2). In an even more specific embodiment, the score is the percentage of amino acid sequence identity between the amino acid sequences of two n-amino acid windows compared. For example, if two 6-amino acid windows have the same amino acid at positions 1, 2, and 3 and the amino acids at positions 4, 5, and 6 differ, the amino acid identity between the two sequences is 50%.

In certain embodiments, the score is a linear function of the number of identical amino acid positions. In other embodiments, the score is an exponential or a logarithmic function of the number of the identical amino acids. The sequences of two n-amino acid windows can be aligned with or without the introduction of gaps. In a specific embodiment, gaps are introduced to maximize the score. In certain embodiments, a penalty is subtracted from the score for each gap. The scoring algorithm can be adjusted to increase the sensitivity of the methods of the invention.

In certain embodiments, the introduction of gaps in either strand of the two amino acid sequences that are being compared with each other is permitted. In a more specific embodiment, a single amino acid gap is introduced between any neighboring amino acids in either one of the two sequences that are being compared with each other. A penalty score worth a percentage of a match is subtracted from the overall alignment score. The better of the ungapped alignment and gapped alignment score for the sliding window is taken for future computation.

In other embodiments, the score depends on the degree of sequence similarity between the two sequences in a sequence comparison. Sequence similarity between amino acid sequences in a sequence comparison can be evaluated using any amino acid substitution matrix known to the skilled artisan. Based on the amino acid substitution matrix, values are assigned to each amino acid substitution between the sequences. In a specific embodiment, higher values are assigned if structural and/or functional properties of the amino acids' side-chains are similar or identical to each other. Highest values are assigned if the amino acids are identical. Structural and/or functional properties of the amino acids' side-chains are similar, e.g., if an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In specific embodiments, an amino acid substitution matrix that can be used with the methods of the invention is the PAM matrix (see, e.g., Dayhoff, Schwartz and Orcutt, 1978, A model of evolutionary change in proteins. Matrices for detecting distant relationships. In Atlas of protein sequence and structure, (Dayhoff, M. O., ed.), vol. 5, pp. 345-358. National biomedical research foundation Washington D.C.).

In specific embodiments, each conserved amino acid exchange at a given position in the n-amino acid window increases the score by the same value as an identical amino acid would. In other embodiments, a conserved amino acid increases the score by a certain percentage of the increase of the score per identical amino acid. In certain embodiments, the percentage can be between 10% and 20%, between 20% and 30%, between 30% and 40%, between 50% and 60%, between 60% and 70%, between 70% and 80%, and between 80% and 90%. In an exemplary embodiment, the percentage is 50%; thus, if between 6-amino acid windows positions 1, 2 and 3 are identical and the amino acids at positions 4, 5 and 6 are conserved amino acid exchanges, the score is 50% (for the identical amino acids) plus half of 50% (for the conserved amino acid exchanges)=75% (or 0.75). Exemplary conserved amino acid exchanges include the exchange of an amino acid with a basic side chain for another amino acid with a basic side chain (e.g., lysine for arginine); exchange of an amino acid with an acidic side chain for another amino acid with an acidic side chain (e.g., aspartic acid for glutamic acid); exchange of an amino acid with an uncharged polar side chain for another amino acid with an uncharged polar side chain (e.g., asparagine for glutamine); and exchange of an amino acid with a nonpolar side chain for another amino acid with a nonpolar side chain (e.g., alanine for valine). In certain embodiments, individual percentages are used for each possible amino acid exchange. In these embodiments, the percentage for a particular amino acid exchange depends on how much the antigenicity is preserved in a peptide following the amino acid exchange. The more the antigenicity is preserved despite the amino acid exchange the higher is the percentage.

In certain embodiments, any other information relating to the accessibility of an n-amino acid window in the protein by an antibody or to the antigenicity of an amino acid sequence may optionally figure into the score. Such information can be obtained for example, but not limited, from structural prediction software programs, experimental structure determination (e.g., x-ray crystallography or Circular Dichroism), or hydrophilicity plots. In certain embodiments, the score is increased by a percentage or a determined value is added if the n-amino acid window is predicted to be on the surface of the protein. In certain embodiments, if the antibody binds to the target protein under denaturing conditions, the predicted location of the n-amino acid window in the folded protein is not figured into the score. Without being bound by theory, under denaturing conditions the natural conformation of the protein is destroyed and epitopes that are buried inside the properly folded protein may be presented on its surface under denaturing conditions. Thus, if the antibody binds to the target protein only under denaturing conditions, the fact that the region is located on the surface of the folded protein is not factored into the score.

In certain embodiments, information about the antigenicity of the n-amino acid window can be figured into the score. Prediction of protein antigenic determinants from amino acid sequences can be obtained by the method of Hopp and Woods (1981, Proc. Natl. Acad. Sci. USA 86:152-156) or the method of Kolaskar and Tongaonkar, (1990, FEBS Letters 276:172-174; see, e.g., homepage of EMBOSS (The European Molecular Biology Open Software Suite)).

In certain embodiments, the more homologous the amino acid sequences of two n-amino acid windows are the higher the score for their comparison is. Thus, the score for an n-amino acid window comparison is positively correlated with the degree of sequence identity or degree of sequence similarity between the n-amino acid windows in the target protein and the cross-reactive protein; and one or more of the following factors (i) the probability that the n-amino acid window is on the surface of the protein; and (ii) the predicted antigenicity of the amino acid sequence of the n-amino acid window (the antigenic index; as predicted by, e.g., Kolaskar and Tongaonkar, (1990, FEBS Letters 276:172-174; see, e.g., homepage of EMBOSS (The European Molecular Biology Open Software Suite)).

In certain other embodiments, the more homologous the amino acid sequences of two n-amino acid windows are the lower is the score for their comparison. Thus, the score for an n-amino acid window comparison is negatively correlated with the sequence identity or sequence similarity between the n-amino acid windows in the target protein and the cross-reactive protein; and one or more of the following factors (i) the probability that the n-amino acid window is on the surface of the protein; and (ii) the predicted antigenicity of the amino acid sequence of the n-amino acid window.

The steps of the methods of the invention are described as if the score for an n-amino acid window is positively correlated with sequence similarity or sequence identity. The skilled artisan would recognize, however, that the methods could be performed in the same way if the score for an n-amino acid window is negatively correlated with the sequence similarity or sequence identity simply by reversing the sign/polarity. E.g., instead of identifying the region of the target protein with the maximum average highest score, the region of the target protein with the minimum average lowest score would have to be identified.

Without being bound by theory, post-translational modifications of a protein can alter the antigenic properties of the protein. Post-translational modifications include, but are not limited to, phoshorylation, glycosylation, myristoylation, acylation, methylation, sulfation, prenylation, vitamin C-dependent modifications (e.g., proline and lysine hydroxylations and carboxy terminal amidation), vitamin K-dependent modification (e.g., carboxylation of glutamine residues), and incorporation of selenocysteine.

In certain embodiments, post-translational modifications are considered in assigning a score. Post-translational modifications are considered if the target and the cross-reactive proteins are expressed in an expression system that supports post-translational modification. In a specific embodiment, binding of the antibody to the target protein is known to depend on post-translational modification. In this embodiment, cross-reactive proteins should be identified from a population of proteins that were expressed in an expression system that supports the type of post-translational modification that is known to be required for binding of the antibody to the target protein. Certain post-translational modifications occur at specific consensus sites in the protein. If the n-amino acid window of the target protein and the n-amino acid window of the cross-reactive protein that are compared with each other have such a consensus sequence in common, the score may be increased (if the score is positively correlated with homology between the amino acid sequences of the n-amino acid windows) by a determined value or percentage.

In certain embodiments, a post-translational modification interferes with the binding of the antibody to the target protein and/or the cross-reactive protein. In such a case, the absence of the consensus sequence for the post-translational modification may be reflected in the value of the score for each n-amino acid window comparison.

In certain embodiments, to determine the degree of sequence identity and/or similarity of two amino acid sequences or of two nucleic acid sequences, the sequences are aligned for optimal comparison purposes (e.g., gaps can be introduced in the sequence of either one of the sequences being compared). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. In a specific embodiment, the degree of identity is expressed as percentage identity. The percentage identity between the two sequences is a function of the number of identical positions shared by the sequences (i.e., % identity=number of identical overlapping positions/total number of positions×100%). In one embodiment, the two sequences are the same length.

In certain embodiments, the determination of sequence identity and/or similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. To obtain gapped alignments for comparison purposes, Gapped BLAST can be utilized as described in Altschul et al., 1997, Nucleic Acids Res. 25:3389-3402. In certain embodiments, when utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. In a specific embodiment, when utilizing the ALIGN program for comparing amino acid sequences, a PAM120 weight residue table, a gap length penalty of 12, and a gap penalty of 4 can be used.

In certain embodiments, the percentage identity between two sequences can be determined using techniques similar to those described above, with or without allowing gaps. In calculating percentage identity, typically only exact matches are counted.

### 5.1.2 Amino Acid Sequence Comparison

In certain embodiments, to determine the score for the amino acid sequences of two n-amino acid windows, the two sequences are aligned with each other. Any method known to the skilled artisan can be used to align the amino acid sequences of two n-amino acid windows. In certain embodiments, aligning two amino acid sequences is matching each amino acid position of the two amino acid position.

In certain embodiments, the amino acid identity or similarity between the amino acid sequences of two n-amino acid windows represents the degree (e.g., the percentage) of amino acid positions at which both n-amino acid windows have the same or a conserved amino acid without the introduction of gaps in one of the sequences. In other embodiments, the introduction of gaps is allowed to maximize the score. In a specific embodiment, no gap penalty is subtracted from the score. In other embodiments, a gap penalty is subtracted from the score for each gap introduced to maximize the score for a particular alignment of two sequences.

In certain embodiments, the introduction of gaps in either strand of the two amino acid sequences that are being compared with each other is permitted. In a more specific embodiment, a single amino acid gap is introduced between any neighboring amino acids in either one of the two sequences that are being compared with each other. A penalty score worth a percentage of a match is subtracted from the overall alignment score. The better of the ungapped alignment and gapped alignment score for the sliding window is taken for future computation.

In certain embodiments, the n-amino acid windows compared are of equal length. In certain other embodiments, the n-amino acid windows compared have different lengths. In certain more specific embodiments, the n-amino acid windows from the target protein are longer than the n-amino acid windows from the cross-reactive proteins. In other embodiments, the n-amino acid windows from the target protein are shorter than the n-amino acid windows from the cross-reactive proteins.

### 5.2 Prediction of Epitopes Bound by a Molecule Other than an Antibody

In certain embodiments, the methods of the invention are used to determine a binding site in a protein that is bound by a molecule other than an antibody. Such a molecule can be a protein, a peptide, a polypeptide, a small organic molecule, a sugar, a polysaccharide, a lipid or an inorganic molecule. In a specific embodiment, the molecule other than an antibody is a drug. In another specific embodiment, the molecule other than an antibody is a nucleic acid. The nucleic acid can be single-stranded or double-stranded, DNA or RNA. In certain specific embodiments, the nucleic acid is at least 3, 5, 7, 10, 15, 20, 25, 30, 35, 40, 45, 50, 60, 65, 70, 75, 80, 90, 95, 100, 150, 200 or at least 250 nucleotides long. In certain specific embodiments, the nucleic acid is at most 3, 5, 7, 10, 15, 20, 25, 30, 35, 40, 45, 50, 60, 65, 70, 75, 80, 90, 95, 100, 150, 200 or at most 250 nucleotides long. In certain embodiments, the nucleic acid is of a specific nucleotide sequence. In a specific embodiment, the nucleic acid sequence is derived from a cis-regulatory sequence of a gene of interest. In a more specific embodiment, cis-regulatory sequence of a gene of interest is known to be bound by a particular transcription factor, in which case the transcription factor is the target protein and the methods of the invention can be used to identify the region of the transcription factor that bind to DNA.

In general, the methods described in section 5.1 for the identification of an epitope that is bound by an antibody can be used for the identification of a binding site in a target protein that is bound by a molecule other than an antibody. Cross-reactive proteins that can be bound by the same molecule as the target molecule can be identified by any method known to the skilled artisan. See section 5.3 for exemplary methods to identify cross-reactive proteins.

In certain embodiments, the molecule is detectably labeled and used to screen a protein array to identify cross-reactive proteins. In other exemplary embodiments, the molecule is linked to a defined moiety, such as, but not limited to, a biotin. The molecule that is linked to the defined moiety is then incubated with a population of proteins under conditions conducive to the formation of complexes between the molecule and any cross-reactive proteins. The complexes are subsequently isolated by virtue of the moiety (e.g., biotin) and the cross-reactive protein is identified by any method known in the art, such as but not limited to, mass spectrometry.

Once the cross-reactive proteins are identified, the binding site(s) can be predicted as described in section 5.1 for epitopes that are bound by an antibody. In certain embodiments, if the molecule is a small molecule, the n-amino acid window is at least 4 amino acids or at most 25 amino acids.

In certain embodiments, the affinity of a molecule to the target protein and to the cross-reactive protein is considered to determine which cross-reactive proteins should be used with the methods of the invention for the prediction of an epitope. In certain embodiments, only cross-reactive proteins that are bound by the molecule with an affinity of at least 10⁻⁴, 10⁻³, 10⁻², or at least 10⁻¹ times the molecule's affinity to the target protein are used with the methods of the invention. In certain embodiments, the molecule binds to the binding site with a binding affinity of at least 1 M⁻¹, 10 M⁻¹, 10² M⁻¹, 10³ M⁻¹, 10⁴ M⁻¹, 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, 10¹⁴ M⁻¹, or at least 10¹⁵ M⁻¹. The affinity of a molecule to a protein can be determined by any method known to the skilled artisan.

### 5.3 Identification of Cross-Reactive Proteins

In certain embodiments of the invention, proteins are identified or are known that can be bound by the same molecule, e.g., an antibody, as the molecule that binds to a target protein. Such cross-reactive proteins can be identified by any method known to the skilled artisan. In certain embodiments, the cross-reactive proteins are from the same species as the target protein. In other embodiments, the cross-reactive proteins are from a species different from the species from which the target protein is derived. In certain embodiments, all cross-reactive proteins are derived from the same species. In other embodiments, the cross-reactive proteins can be derived from different species. In certain embodiments, the target protein is derived from bacteria, yeast, insects, humans, and/or non-human mammals such as mice, rats, cats, dogs, pigs, cows and horses. In certain embodiments, the cross-reactive protein is derived from bacteria, yeast, insects, humans, and/or non-human mammals such as mice, rats, cats, dogs, pigs, cows and horses.

In certain embodiments, a protein array is screened with the molecule or the antibody that binds to the target protein to identify cross-reactive proteins. In certain embodiments, the antibody or the molecule is detectably labeled and incubated with the protein array under conditions conducive to binding of the molecule to the proteins of the protein array. Subsequently, the protein array is washed to remove any unspecifically bound antibodies or molecules, respectively. After the washing step, the proteins that are bound by the antibody or the molecule, respectively, are identified by virtue of the label. If the protein array is a positionally addressable array, the proteins that can be bound by the antibody or molecule can be identified via their location on the microarray. If the microarray is not positionally addressable, the proteins can be identified by any method known to the skilled artisan, such as, but not limited to, microsequencing, sequencing of the nucleic acid that encodes the protein, or mass spectrometry.

In certain embodiments, cross-reactive proteins are identified under denaturing conditions. In other embodiments, cross-reactive protein are identified under non-denaturing conditions. Cross-reactive protein can be identified by any method known to the skilled artisan, such as, but not limited to, immunoprecipitation, Western blot analysis, or affinity chromatography.

If an epitope that is specifically bound by an antibody is to be identified, any method known to the skilled artisan can be used to identify proteins that are also specifically bound by the antibody. In certain embodiments, the antibody is incubated with a plurality of proteins under conditions conducive to the binding between cross-reactive protein and antibody. Subsequently, the antibody-cross-reactive protein complexes are isolated and the cross-reactive proteins are identified. In certain embodiments, the antibody is linked to a solid substrate and incubated with a plurality of proteins under conditions conducive to the binding between cross-reactive protein and antibody. Subsequently, the solid substrate is washed to remove any unspecifically bound protein. The cross-reactive proteins can subsequently be removed from the solid substrate-antibody-cross-reactive protein complexes and identified by any method known to the skilled artisan. Exemplary methods for the identification of proteins include, but are not limited to, mass-spectrometry and microsequencing. In other embodiments, a plurality of known proteins with known molecular weights are subjected to Western blot analysis with the antibody. The cross-reactive proteins can then be identified by virtue of their molecular weights.

If a binding site in a protein that is bound by a particular molecule is to be identified, any method known to the skilled artisan can be used to identify any cross-reactive proteins. In certain embodiments, the molecule is incubated with a plurality of proteins under conditions conducive to the binding between the molecule and the cross-reactive proteins. Subsequently, the molecule-cross-reactive protein complexes are purified by any method known to the skilled artisan. In a specific embodiment, the molecule-cross-reactive protein complexes are purified using an antibody that recognizes and binds to the molecule. In other embodiments, the molecule is linked to a defined moiety, such as, but not limited to, biotin. The molecule that is linked to the defined moiety is then incubated with a population of proteins under conditions conducive to the formation of complexes between the molecule and any cross-reactive proteins. The complexes are subsequently isolated by virtue of the moiety (e.g., biotin) and the cross-reactive protein is identified by any method known in the art, such as but not limited to, mass spectrometry and microsequencing.

Post-translation modification of a proteins is a factor to be considered in identifying the cross-reactive proteins. Whether the binding of the antibody or the molecule other than an antibody to the target molecule is dependent on any modification of the target protein, such as, but not limited to, phosphorylation, glycosylation or the addition of lipids, can be determined by any method known in the art. In an exemplary embodiment, the post-translational modification can be removed from the target protein by any method known to the skilled artisan. Once the post-translational modification is removed, the antibody or the molecule other than an antibody is tested for binding to the target protein by any method known in the art. If the antibody or the molecule other than an antibody binds to the target protein in the absence of the post-translational modification, the post-translational modification is not essential for binding of the antibody or the molecule other than an antibody to the target protein. In certain embodiments, the post-translational modification is removed from the target protein enzymatically, e.g., phosphate can be removed from the target protein by incubation of the target protein with phosphatase. Without being bound by theory, it is preferred that the binding of the antibody or the molecule other than an antibody bind to the target protein independent of a post-translational modification because the methods for epitope prediction of the present invention are based on primary amino acid sequence comparison.

In certain embodiments, the affinity of an antibody or a molecule other than an antibody to the target protein and to the cross-reactive protein is considered to determine which cross-reactive proteins should be used with the methods of the invention for the prediction of an epitope. In certain embodiments, only cross-reactive proteins that are bound by the antibody or the molecule with an affinity of at least 10⁻⁴, 10⁻³, 10⁻², or at least 10⁻¹ times the antibody's affinity to the target protein are used with the methods of the invention. In certain embodiments, the antibody or the molecule binds to the epitope or the binding site, respectively, with a binding affinity of at least 1 M⁻¹, 10 M⁻¹, 10² M⁻¹, 10³ M⁻¹, 10⁴ M⁻¹, 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, 10¹⁴ M⁻¹, or at least 10¹⁵ M⁻¹. The affinity of an antibody to a protein can be determined by any method known to the skilled artisan. Exemplary methods are described in section 5.8. In other embodiments, any cross-reactive protein identified is used with the methods of the invention.

### 5.3.1 Screening of Protein Arrays

In certain embodiments, any protein array can be used with the methods of the present invention to identify cross-reactive proteins. The protein arrays can be screened with an antibody against a target protein to identify cross-reactive proteins that are also bound by the antibody. The arrays can also be screened with a molecule other than an antibody that binds to a target molecule to identify cross-reactive proteins that are also bound by the molecule. In certain embodiments, the protein chip is a positionally addressable array of proteins.

Cross-reactive proteins on the chip are identified by incubating a protein chip with the antibody under conditions conducive to binding between a cross-reactive protein and the antibody. In certain embodiments, the incubation step is followed by a washing step to remove any unspecifically bound antibodies. Without being bound by theory, the stringency of the washing step affects the number of the identified cross-reactive proteins. If the stringency is high, only the cross-reactive proteins with the highest affinity to the antibody are identified. If the stringency is lower, more cross-reactive proteins are identified. The stringency of the washing step depends on several parameters, such as, but not limited to, salt concentration. The cross-reactive protein can be detected using standard detection assays such as luminescence, chemiluminescence, fluorescence or chemifluorescence. For example, the antibody against the target protein that also binds to a cross-reactive protein on the protein chip is recognized by a fluorescently labeled secondary antibody, which is then measured with an instrument (e.g., a Molecular Dynamics scanner) that excites the fluorescent product with a light source and detects the subsequent fluorescence. For greater sensitivity, a primary antibody to the protein of interest is recognized by a secondary antibody that is conjugated to an enzyme such as alkaline phosphatase or horseradish peroxidase. In the presence of a luminescent substrate (for chemiluminescence) or a fluorogenic substrate (for chemifluorescence), enzymatic cleavage yields a highly luminescent or fluorescent product which can be detected and quantified by using, for example, a Molecular Dynamics scanner. Alternatively, the signal of a fluorescently labeled secondary antibody can be amplified using an alkaline phosphatase-conjugated or horseradish peroxidase-conjugated tertiary antibody.

In other embodiments, a protein chip is screened with a molecule other than an antibody to identify cross-reactive proteins that also bind to the molecule. Cross-reactive proteins on the chip are identified by incubating the protein chip with the molecule under conditions conducive to binding between a cross-reactive protein and the antibody. In certain embodiments, the incubation step is followed by a washing step to remove any unspecifically bound molecules. Without being bound by theory, the stringency of the washing step affects the number of the identified cross-reactive proteins. If the stringency is high, only the cross-reactive proteins with the highest affinity to the antibody are identified. If the stringency is lower, more cross-reactive proteins are identified. The stringency of the washing step depends on several parameters, such as, but not limited to, salt concentration. The cross-reactive protein can be detected using standard detection assays such as luminescence, chemiluminescence, fluorescence or chemifluorescence. For example, the molecule that binds to the target protein and that also binds to a cross-reactive protein on the protein chip is recognized by a fluorescently labeled antibody, which is then measured with an instrument (e.g., a Molecular Dynamics scanner) that excites the fluorescent product with a light source and detects the subsequent fluorescence. For greater sensitivity, a primary antibody to the molecule of interest is recognized by a secondary antibody that is conjugated to an enzyme such as alkaline phosphatase or horseradish peroxidase. In the presence of a luminescent substrate (for chemiluminescence) or a fluorogenic substrate (for chemifluorescence), enzymatic cleavage yields a highly luminescent or fluorescent product which can be detected and quantified by using, for example, a Molecular Dynamics scanner. Alternatively, the signal of a fluorescently labeled secondary antibody can be amplified using an alkaline phosphatase-conjugated or horseradish peroxidase-conjugated tertiary antibody. In other embodiments, the molecule is linked to a moiety that can be bound by a detectably labeled antibody. Any other method known in the art to detect the molecule once bound to a cross-reactive protein on the protein chip can be used with the methods of the invention.

In a specific embodiment, a protein array that can be used to identify cross-reacting proteins comprises a plurality of potential antigens on a solid support, with each different antigen being at a different position on the solid support, wherein the density of different antigens is at least 100 different antigens per cm², and detecting positions on the solid support where binding by an antibody in the antibody preparation occurs. The antibody preparation can be, but is not limited to, Fab fragments, antiserum, and polyclonal, monoclonal, chimeric, single chain, humanized, or synthetic antibodies. For example, an antiserum can be characterized by screening disease-specific, tissue-specific, or other identified collections of antigens, and determining which antigens are recognized. In a specific embodiment, protein chip arrays have similar or related antigens.

The protein chips to be used with the methods of the present invention are not limited in their physical dimensions and may have any dimensions that are convenient. For the sake of compatibility with current laboratory apparatus, protein chips the size of a standard microscope slide or smaller are preferred. Most preferred are protein chips sized such that two chips fit on a microscope slide. Also preferred are protein chips sized to fit into the sample chamber of a mass spectrometer.

In certain embodiments, a protein chip that can be used with the methods of the present invention comprises a flat surface, such as, but not limited to, glass slides or nitrocellulose-coated glass slides. Dense protein arrays can be produced on, for example, glass slides, such that chemical reactions and assays can be conducted, thus allowing large-scale parallel analysis. Proteins or probes are bound covalently or non-covalently to the flat surface of the solid support. The proteins or probes can be bound directly to the flat surface of the solid support, or can be attached to the solid support through a linker molecule or compound. The linker can be any molecule or compound that derivatizes the surface of the solid support to facilitate the attachment of proteins or probes to the surface of the solid support. The linker may covalently or non-covalently bind the proteins or probes to the surface of the solid support. In addition, the linker can be an inorganic or organic molecule. Preferred linkers are compounds with free amines. Other preferred linkers are compounds with free thiols. In a specific embodiment, the linker is 3-glycidooxypropyltrimethoxysilane (GPTS).

Proteins can be spotted on the protein chips as fusion proteins, in which a defined domain is attached to one of a variety of natural proteins, or can be intact non-fusion proteins.

In another embodiment, protein-containing cellular material, such as but not limited to vesicles, endosomes, subcellular organelles, and membrane fragments, can be placed on the protein chip (e.g., in wells) to identifv cross-reactive proteins. In another embodiment, a whole cell is placed on the protein chip (e.g., in wells). In a further embodiment, the protein, protein-containing cellular material, or whole cell is attached to the solid support of the protein chip.

The protein can be purified prior to placement on the protein chip or can be purified during placement on the chip via the use of reagents that bind to particular proteins, which have been previously placed on the protein chip. Partially purified protein-containing cellular material or cells can be obtained by standard techniques (e.g., affinity or column chromatography) or by isolating centrifugation samples (e.g., P1 or P2 fractions).

Furthermore, proteins, protein-containing cellular material, or cells can be embedded in artificial or natural membranes prior to or at the time of placement on the protein chip. In another embodiment, proteins, protein-containing cellular material, or cells can be embedded in extracellular matrix component(s) (e.g., collagen or basal lamina) prior to or at the time of placement on the protein chip. The proteins can be in solution, or bound to the surface of the solid support (e.g., in a well, or on a flat surface), or bound to a substrate (e.g., bead) placed in a well of the solid support.

Protein chips on which proteins are embedded in membranes, e.g., vesicles, can be particularly useful for identifying cross-reactive proteins if the conformation of the protein depends on the association of the protein with the membrane. Similarly, protein chips on which the proteins are embedded in extracellular matrix material can be particularly useful for identifying cross-reactive proteins if the conformation of the protein depends on its association with the extracellular matrix. Without being bound by theory, the conformation of the protein, and in particular the conformation of the epitope of interest determines its antigenicity.

In certain embodiments, a protein chip used for the identification of cross-reactive proteins has wells. The placement of proteins in wells can be accomplished by using any dispensing means, such as bubble jet or ink jet printer heads. A micropipette dispenser is preferred. The placement of proteins can either be conducted manually or the process can be automated through the use of a computer connected to a machine. Proteins can be bound to a substrate (e.g., beads) that is placed in the wells. Other substrates include, but are not limited to, nitrocellulose particles, glass beads, plastic beads, magnetic particles, and latex particles. Alternatively, the proteins or probes are bound covalently or non-covalently to the surface of the solid support in the wells. The proteins or probes can be bound directly to the surface of the solid support (in the well), or can be attached to the solid support through a linker molecule or compound. The linker can be any molecule or compound that derivatizes the surface of the solid support to facilitate the attachment of proteins or probes to the surface of the solid support. The linker may covalently bind the proteins or probes to the surface of the solid support or the linker may bind via non-covalent interactions. In addition, the linker can be an inorganic or organic molecule. Preferred linkers are compounds with free amines. In a specific embodiment, the linker is 3-glycidooxypropyltrimethoxysilane (GPTS).

Proteins which are non-covalently bound to the well surface may utilize a variety of molecular interactions to accomplish attachment to the well surface such as, for example, hydrogen bonding, van der Waals bonding, electrostatic, or metal-chelate coordinate bonding. Further, DNA-DNA, DNA-RNA and receptor-ligand interactions are types of interactions that utilize non-covalent binding. Examples of receptor-ligand interactions include interactions between antibodies and antigens, DNA-binding proteins and DNA, enzyme and substrate, avidin (or streptavidin) and biotin (or biotinylated molecules), and interactions between lipid-binding proteins and phospholipid membranes or vesicles. For example, proteins can be expressed with fusion protein domains that have affinities for a substrate that is attached to the surface of the well. Suitable substrates for fusion protein binding include trypsin/anhydrotrypsin, glutathione, immunoglobulin domains, maltose, nickel, or biotin and its derivatives, which bind to bovine pancreatic trypsin inhibitor, glutathione-S-transferase, antigen, maltose binding protein, poly-histidine, chitin binding domain (for the binding to chitin) and avidin/streptavidin, respectively. In certain, more specific embodiments, the poly-histidine domain consists of six histidines (e.g., a HisX6 tag).

The protein arrays that can be used to identify cross-reactive proteins have spots of full-length proteins, portions of full-length proteins, and/or peptides whether prepared from recombinant overexpression in an organism, produced via fragmentation of larger proteins, or chemically synthesized. Protein arrays with proteins from bacteria, yeast, insects, humans, and/or non-human mammals such as mice, rats, cats, dogs, pigs, cows and horses, can be used to identify cross-reactive proteins. Further, fusion proteins in which a defined domain is attached to one of a variety of natural or synthetic proteins can be utilized. Proteins used in this invention can be purified prior to being attached to the surface of a solid support, or deposited into, the wells of the protein chip, or purified during attachment via the use of reagents which have been previously attached to, or deposited into, the wells of the protein chip. These reagents include those that specifically bind proteins in general, or bind to a particular group of proteins. Proteins can be embedded in artificial or natural membranes (e.g., liposomes, membrane vesicles) prior to, or at the time of attachment to the protein chip. Alternatively, the proteins can be delivered into the wells of the protein chip.

Proteins used for the preparation of protein chips that can be used with the methods of the present invention are preferably expressed by methods known in the art. The InsectSelect system from Invitrogen (Carlsbad, Calif., catalog no. K800-01), a non-lytic, single-vector insect expression system that simplifies expression of high-quality proteins and eliminates the need to generate and amplify virus stocks, is a preferred expression system. The preferred vector in this system is pIB/V5-His TOPO TA vector (catalog no. K890-20). Polymerase chain reaction (PCR) products can be cloned directly into this vector, using the protocols described by the manufacturer, and the proteins are then expressed with N-terminal histidine (His) labels which can be used to purify the expressed protein.

The BAC-TO-BAC™ system, another eukaryotic expression system in insect cells, available from Lifetech (Rockville, Md.), is also a preferred expression system. Rather than using homologous recombination, the BAC-TO-BAC™ system generates recombinant baculovirus by relying on site-specific transposition in E. coli. Gene expression is driven by the highly active polyhedrin promoter, and therefore can represent up to 25% of the cellular protein in infected insect cells.

Post-translational modification of proteins is a consideration in selecting the expression system. If the binding of the molecule, e.g., antibody, to the target protein is dependent on post-translational modification of the target protein, it is preferred that the population of proteins among which the cross-reactive proteins are identified is expressed in an expression system that supports post-translational modification.

In certain embodiments, the proteins to be placed on protein microarrays for the identification of cross-reactive proteins comprise a first tag and a second tag. The advantages of using double-tagged proteins include the ability to obtain highly purified proteins, as well as providing a streamlined manner of purifying proteins from cellular debris and attaching the proteins to a solid support. In a particular embodiment, the first tag is a glutathione-S-transferase tag (“GST tag”) and the second tag is a poly-histidine tag (“His tag”). In a further embodiment, the GST tag and the His tag are attached to the amino-terminal end of the protein or the substrate. Alternatively, the GST tag and the His tag are attached to the carboxy-terminal end of the protein or substrate.

In certain embodiments, a protein is expressed as a fusion protein with a chitin binding domain in combination with another tag, such as a GST tag or a His tag. In other embodiments, a protein is expressed as a fusion protein with a chitin binding domain and an intein. In a more specific embodiment, the proteins and/or substrates are expressed using the IMPACT™-CN system from New England Biolabs Inc.

In yet another embodiment, the GST tag is attached to the amino-terminal end of the protein or substrate. In a further embodiment, the His tag is attached to the carboxy-terminal end of the protein or substrate. In yet another embodiment, the His tag is attached to the amino-terminal end of the protein or substrate. In a further embodiment, the GST tag is attached to the carboxy-terminal end of the protein or substrate.

In yet another embodiment, the protein or substrate comprises a GST tag and a His tag, and neither the GST tag nor the His tag is located at the amino-terminal or carboxy-terminal end of the protein. In a specific embodiment, the GST tag and His tag are located within the coding region of the protein or substrate of interest; preferably in a region of the protein not affecting the enzymatic activity of interest and preferably in a region of the substrate not affecting the suitability of the substrate to be modified by the enzymatic reaction of interest.

In one embodiment, the first tag is used to purify a fusion protein. In another embodiment, the second tag is used to attach a fusion protein to a solid support. In a specific further embodiment, the first tag is a GST tag and the second tag is a His tag.

A binding agent that can be used to purify a protein or a substrate can be, but is not limited to, a glutathione bead, a nickel-coated solid support, and an antibody. In one embodiment, the complex comprises a fusion protein having a GST tag bound to a glutathione bead. In another embodiment, the complex comprises the a fusion protein having a His tag bound to a nickel-coated solid support. In yet another embodiment, the complex comprises the protein of interest bound to an antibody and, optionally, a secondary antibody.

### 5.4 Methods that can be Used in Combination with the Methods of the Invention

The methods of the present invention can be optionally combined with any method known in the art to predict or determine antigenic sites, epitopes, and binding sites in a protein. The results of such other techniques can be factored in the results of the present method at different steps of the methods of the invention. Levels of the methods of the present invention where results of such other techniques can be factored in include, but are not limited to, selection of regions in the target protein to be used with the methods of the invention, assignment of scores (see also section 5.1.1), and selection of the binding site among regions with the highest average scores.

Prediction techniques that can be used optionally in combination with the methods of the invention include, but are not limited to, the antigenic index of the n-amino acid window as determined by the method of Hopp and Woods (1981, Proc. Natl. Acad. Sci. USA 86:152-156) and the method of Kolaskar and Tongaonkar, (1990, FEBS Letters 276:172-174; see, e.g., European Molecular Biology Open Software Suite (“EMBOSS”) webpage).

Software programs that predict the three-dimensional structure of the folded protein or software programs that predict whether a particular region of a protein is on the surface of the protein or buried inside the protein based on the hydrophilicity of the sidechains of the amino acids in that region can be used in combination with the methods of the invention. In combining the methods of the invention with such prediction programs, it is important to consider under which conditions, i.e., non-denaturing or denaturing, the target protein is bound by the molecule, e.g., the antibody. For example, if the target protein is bound only under non-denaturing conditions, and the program predicts that the candidate epitope is on the surface of the target protein under non-denaturing conditions the candidate epitope is more likely to be the epitope that is bound by the molecule.

The prediction of post-translational modification in a region of the protein. If binding of the molecule, e.g., the antibody, is dependent on post-translational modification, and the n-amino acid window contains the site for such a post-translational modification, this n-amino acid window is more likely than another n-amino acid window that does not contain the site for such a post-translational modification even if the scores for the two n-amino acid windows are equal. In certain embodiments, the score for a particular amino acid sequence comparison is increased by a specific value or multiplied by a specific factor if a consensus sequence for a post-translational modification is present in both amino acid sequences. In a more specific embodiments, the score for a particular amino acid sequence comparison is increased by a specific value or multiplied by a specific factor if the consensus sequence for the post-translational modification that is know to be required for binding by the molecule is present in both amino acid sequences.

Experimental techniques can be used to determine the region or domain of the protein that contains the binding site. Such techniques include the determination of whether fragments or deletion mutants of the target protein are bound by the molecule.

### 5.5 Implementation Systems and Methods

The analytical methods of the present invention for predicting a binding site of a molecule in a protein can preferably be implemented using a computer system, such as the computer system described in this section, according to the following programs and methods. Such a computer system can also preferably store and manipulate measured data obtained in various experiments that can be used by a computer system implemented with the analytical methods of this invention. Accordingly, such computer systems are also considered part of the present invention.

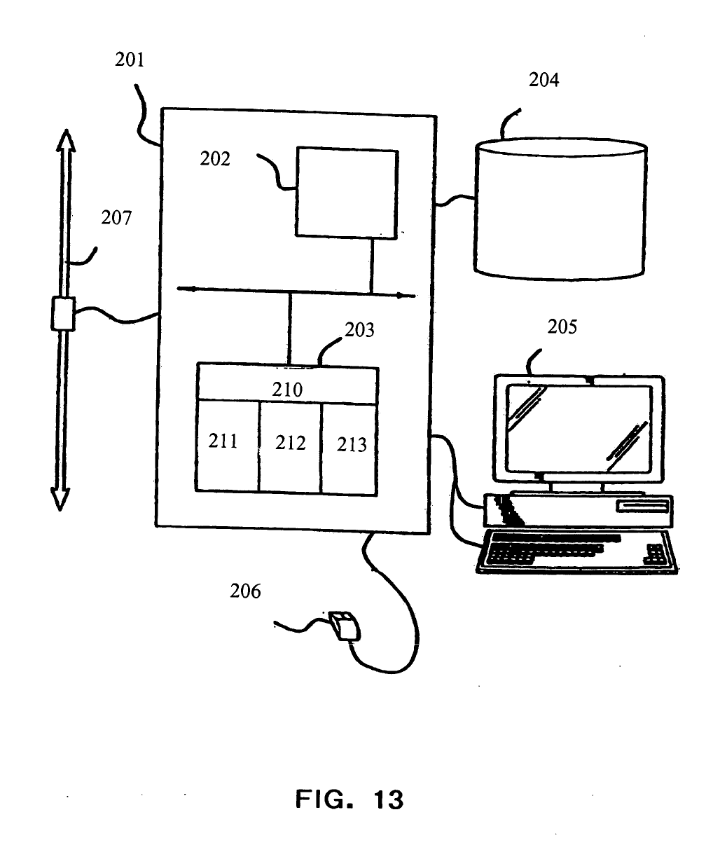

An exemplary computer system suitable from implementing the analytic methods of this invention is illustrated in FIG. 13. Computer system 201 is illustrated here as comprising internal components and as being linked to external components. The internal components of this computer system include one or more processor elements 202 interconnected with a main memory 203. For example, computer system 201 can be an Intel Pentium®-based processor of 200 MHZ or greater clock rate and with 32 MB or more main memory. In a preferred embodiment, computer system 201 is a cluster of a plurality of computers comprising a head “node” and eight sibling “nodes,” with each node having a central processing unit (“CPU”). In addition, the cluster also comprises at least 128 MB of random access memory (“RAM”) on the head node and at least 256 MB of RAM on each of the eight sibling nodes. Therefore, the computer systems of the present invention are not limited to those consisting of a single memory unit or a single processor unit.

The external components can include a mass storage 204. This mass storage can be one or more hard disks that are typically packaged together with the processor and memory. Such hard disk are typically of 1 GB or greater storage capacity and more preferably have at least 6 GB of storage capacity. For example, in a preferred embodiment, described above, wherein a computer system of the invention comprises several nodes, each node can have its own hard drive. The head node preferably has a hard drive with at least 6 GB of storage capacity whereas each sibling node preferably has a hard drive with at least 9 GB of storage capacity. A computer system of the invention can further comprise other mass storage units including, for example, one or more floppy drives, one more CD-ROM drives, one or more DVD drives or one or more DAT drives.

Other external components typically include a user interface device 205, which is most typically a monitor and a keyboard together with a graphical input device 206 such as a “mouse.” The computer system is also typically linked to a network link 207 which can be, e.g., part of a local area network (“LAN”) to other, local computer systems and/or part of a wide area network (“WAN”), such as the Internet, that is connected to other, remote computer systems. For example, in the preferred embodiment, discussed above, wherein the computer system comprises a plurality of nodes, each node is preferably connected to a network, preferably an NFS network, so that the nodes of the computer system communicate with each other and, optionally, with other computer systems by means of the network and can thereby share data and processing tasks with one another.

Loaded into memory during operation of such a computer system are several software components that are also shown schematically in FIG. 13. The software components comprise both software components that are standard in the art and components that are special to the present invention. These software components are typically stored on mass storage such as the hard drive 204, but can be stored on other computer readable media as well including, for example, one or more floppy disks, one or more CD-ROMs, one or more DVDs or one or more DATs. Software component 210 represents an operating system which is responsible for managing the computer system and its network interconnections. The operating system can be, for example, of the Microsoft Windows™ family such as Windows 95, Window 98, Windows NT or Windows 2000. Alternatively, the operating software can be a Macintosh operating system, a UNIX operating system or the LINUX operating system. Software components 211 comprises common languages and functions that are preferably present in the system to assist programs implementing methods specific to the present invention. Languages that can be used to program the analytic methods of the invention include, for example, C and C++, FORTRAN, PERL, HTML, JAVA, and any of the UNIX or LINUX shell command languages such as C shell script language. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

Software component 212 comprises any analytic methods of the present invention described supra, preferably programmed in a procedural language or symbolic package. For example, software component 212 preferably includes programs that cause the processor to implement steps of accepting a plurality of measured data and storing the measured data in the memory. For example, the computer system can accept measured data that are manually entered by a user (e.g., by means of the user interface). More preferably, however, the programs cause the computer system to retrieve measured data from a database. Such a database can be stored on a mass storage (e.g., a hard drive) or other computer readable medium and loaded into the memory of the computer, or the compendium can be accessed by the computer system by means of the network 207.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

### 5.6 Confirmation of Epitopes

In certain embodiments, any method known in the art can optionally be used to confirm that the predicted epitope is the site of the protein that is bound by the antibody or molecule. In certain exemplary embodiments, inhibition of binding between a cross-reactive protein or the target protein and the antibody or the molecule by a peptide that contains the sequence of the epitope is measured. Inhibition of binding between a cross-reactive protein or the target protein and the antibody or the molecule in the presence of a peptide that contains the sequence of the epitope demonstrates that the identified epitope is the site of the protein that is bound by the antibody or the molecule. In other embodiments, the ability of the target protein or a cross-reactive protein in which the predicted epitope has been deleted or mutated to still be bound by the antibody or the molecule is tested. If the antibody or the molecule fails to bind the target protein or a cross-reactive protein in which the predicted epitope has been deleted or mutated, the epitope is the site in the protein that is bound by the antibody or the molecule.

### 5.7 Generation of Antibodies

In certain embodiments of the invention, an antibody is generated against the target protein. Any method known to the skilled artisan can be used to generate antibodies against the target protein. In certain embodiments, the full-length target protein or fragments thereof can be used as immunogen to generate antibodies which immunospecifically bind such immunogen. The binding affinity of an antibody to an antigen, such as the target protein, can be determined by any method known to the skilled artisan. Such antibodies include, but are not limited to, polyclonal, monoclonal, chimeric, single chain, Fab fragments, and an Fab expression library.

In certain embodiments, a peptide that contains an epitope that was predicted with the methods of the invention is used as an immunogen to generate antibodies.

Various procedures known in the art may be used for the production of polyclonal antibodies to a target protein, or a fragment, derivative, or homolog of the target protein.

For production of the antibody, various host animals can be immunized by injection with a target protein, or a fragment or a derivative thereof. Such host animals include, but are not limited to, rabbits, mice, rats, etc. Various adjuvants can be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacille Calmette-Guerin (BCG) and Corynebacterium parvum.

For preparation of monoclonal antibodies directed towards a target protein, or a derivative, fragment, or homolog thereof, any technique that provides for the production of antibody molecules by continuous cell lines in culture may be used. Such techniques include, but are not restricted to, the hybridoma technique originally developed by Kohler and Milstein (1975, Nature 256:495-497), the trioma technique (Gustafsson et al., 1991, Hum. Antibodies Hybridomas 2:26-32), the human B-cell hybridoma technique (Kozbor et al., 1983, Immunology Today 4:72), and the EBV hybridoma technique to produce human monoclonal antibodies (Cole et al., 1985, In: Monoclonal Antibodies and Cancer Therapy, Alan R. Liss, Inc., pp. 77-96). In an additional embodiment, monoclonal antibodies can be produced in germ-free animals utilizing recent technology described in International Patent Application PCT/US90/02545.

Human antibodies may be used with the methods of the present invention and can be obtained by using human hybridomas (Cote et al., 1983, Proc. Natl. Acad. Sci. USA 80:2026-2030) or by transforming human B cells with EBV virus in vitro (Cole et al., 1985, In: Monoclonal Antibodies and Cancer Therapy, Alan R. Liss, Inc., pp. 77-96). Techniques developed for the production of “chimeric antibodies” (Morrison et al., 1984, Proc. Natl. Acad. Sci. USA 81:6851-6855; Neuberger et al., 1984, Nature 312:604-608; Takeda et al., 1985, Nature 314:452-454) by splicing the genes from a mouse antibody molecule specific for the complex together with genes from a human antibody molecule of appropriate biological activity can be used.

Techniques described for the production of single chain antibodies (U.S. Pat. No. 4,946,778) can be used to produce antibodies against a target protein. An additional embodiment of the invention utilizes the techniques described for the construction of Fab expression libraries (Huse et al., 1989, Science 246:1275-1281) to allow rapid and easy identification of monoclonal Fab fragments with the desired specificity for a target protein. Non-human antibodies can be “humanized” by known methods (e.g., U.S. Pat. No. 5,225,539).

Antibody fragments that contain the idiotypes of the target protein can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments.

In the production of antibodies, screening for the desired antibody can be accomplished by techniques known in the art, e.g., ELISA (enzyme-linked immunosorbent assay). To select antibodies specific to a particular domain of the target protein, or a derivative thereof, one may assay generated hybridomas for a product that binds to the fragment of the complex, or a derivative thereof, that contains such a domain.

### 5.8 Determination of Antibody Affinity

In certain embodiments, the affinity of an antibody to the target protein and to the cross-reactive protein is determined. The affinities are useful in determining which cross-reactive proteins should be used with the methods of the invention for the prediction of an epitope. The affinities are also useful for weighting the different scores for the different cross-reactive proteins in calculating the average of the highest scores.

The binding affinity of an antibody (including a scFv or other molecule comprising, or alternatively consisting of, antibody fragments or variants thereof) to an antigen and the off-rate of an antibody-antigen interaction can be determined by competitive binding assays. One example of a competitive binding assay is a radioimmunoassay comprising the incubation of labeled antigen (e.g., ³H or ¹²⁵I with the antibody of interest in the presence of increasing amounts of unlabeled antigen, and the detection of the antibody bound to the labeled antigen. The affinity of the antibody of the present invention and the binding off-rates can be determined from the data by Scatchard plot analysis. Competition with a second antibody can also be determined using radioimmunoassays. In this case, an antigen is incubated with an antibody of the present invention conjugated to a labeled compound (e.g., ³H or ¹²⁵I) in the presence of increasing amounts of an unlabeled second antibody.

Determination of the kinetic parameters of antibody binding can be determined for example by the injection of monoclonal antibody (“mAb”) at varying concentration in buffer over a sensor chip surface, onto which the antigen has been immobilized. In certain embodiments, surface plasmon resonance is used to determine the kinetic parameters of antibody binding.

Once an entire data set is collected, the resulting binding curves are globally fitted using algorithms supplied by the instrument manufacturer, BIAcore, Inc. (Piscataway, N.J.). All data are fitted to a 1:1 Langmuir binding model. These algorithm calculate both the kₒₙ and the k_{off}, from which the apparent equilibrium binding constant, K_{D}, is deduced as the ratio of the two rate constants (i.e. k_{off}/kₒₙ). More detailed treatments of how the individual rate constants are derived can be found in the BIAevaluation Software Handbook (BIAcore, Inc., Piscataway, N.J.).

In certain embodiments, the affinity of an antibody is determined by virtue of the signal intensity obtained from screening a protein array with the antibody. If the proteins on a protein array are all present on the array in approximately equimolar amounts, the signal intensity of an antibody bound to a protein on the array corresponds to the binding affinity of the antibody to the protein.

### 6. EXAMPLES

### Example 1

### Analyzing Antibody specificity with Whole Proteome Microarrays

As an initial test of this approach, a number of polyclonal and monoclonal antibodies against yeast proteins was screened with a yeast proteome microarray and it was found that, in addition to recognizing their cognate proteins (target protein), the antibodies cross-reacted with other yeast proteins (cross-reactive proteins) to varying degrees. Some of the interactions of the antibodies with non-cognate proteins could be deduced by alignment of the primary amino acid sequences of the antigens and cross-reactive protein using a novel algorithm specifically designed for this purpose; however, these interactions could not be predicted a priori without the knowledge of the cross-reactive proteins. The novel sequence analysis algorithm also allows the identification of common epitopes among cross-reactive proteins and the target protein. These findings demonstrate that proteome array technology has enormous potential to improve antibody design/selection for applications in both medicine and research.

Results

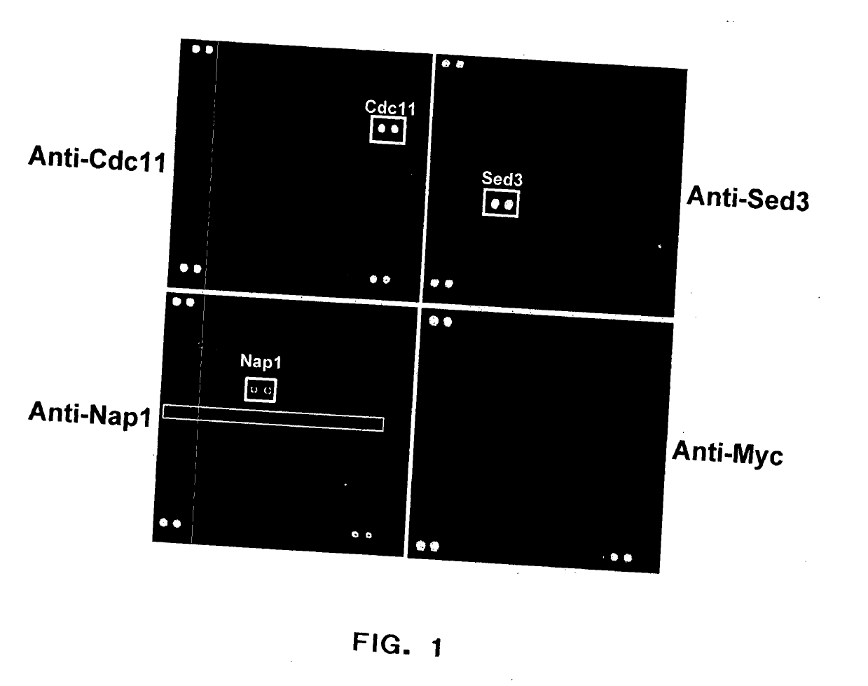

Antibody probing of yeast proteome arrays. The yeast proteome microarrays were probed with a variety of goat and rabbit polyclonal antibodies and mouse monoclonal antibodies prepared against yeast antigens (Table 1). Six of the polyclonal antibodies were generated against peptides and two polyclonal antibodies were prepared against full-length proteins. Five of the eight polyclonal antibodies were affinity purified. Yeast proteome arrays were also probed with three monoclonal antibodies generated against proteins or protein fragments. Three monoclonal antibodies that recognized non-yeast peptides were also used as negative controls. FIG. 1 shows a few examples of the probings carried out in this study, including an example of a probing with one of the monoclonal antibodies that was raised against a non-yeast protein sequence; this figure also demonstrates the excellent signal to noise obtained on these arrays.

Following each antibody probing, the fluorescence intensity of every spot on the array was quantitated, and the number of proteins exhibiting a signal-to-background ratio greater than or equal to 2.0 was scored. As shown in Table 1, the number of reactive proteins varied with the particular antibody. For the anti-peptide polyclonal antibodies, 1 to 9 proteins were observed to give signals on the array, while for the polyclonal antibodies generated against full length proteins 1 to as many as 1770 signals were observed. Finally, 1 to 4 proteins were observed to give signals with the monoclonal antibodies directed against yeast proteins. None of the three control monoclonal antibodies against non-yeast proteins gave signals that were significantly over background.

Analysis of polyclonal antibody cross-reactivity. There are two possible explanations for the cross-reactivity of antibodies with non-cognate yeast proteins on the array—either the positive proteins have an epitope in common with the cognate antigen or the cognate antigen co-purifies with the cross-reacting antigen in the yeast protein preparations. To distinguish between these possibilities, polyclonal antibodies that gave signals with non-cognate proteins on the yeast proteome array were examined further by Western analysis.

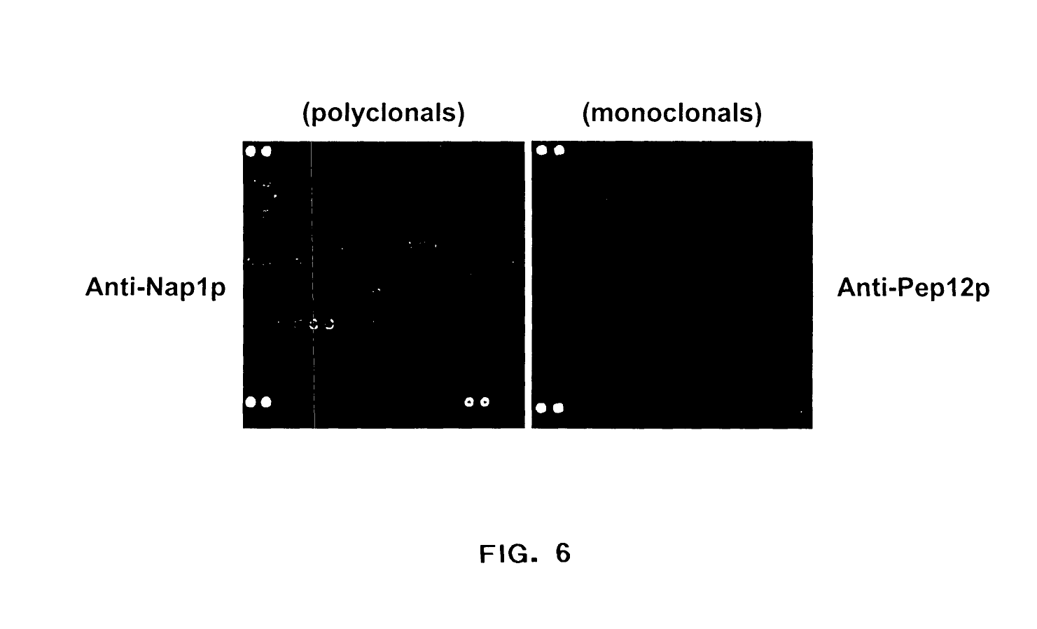

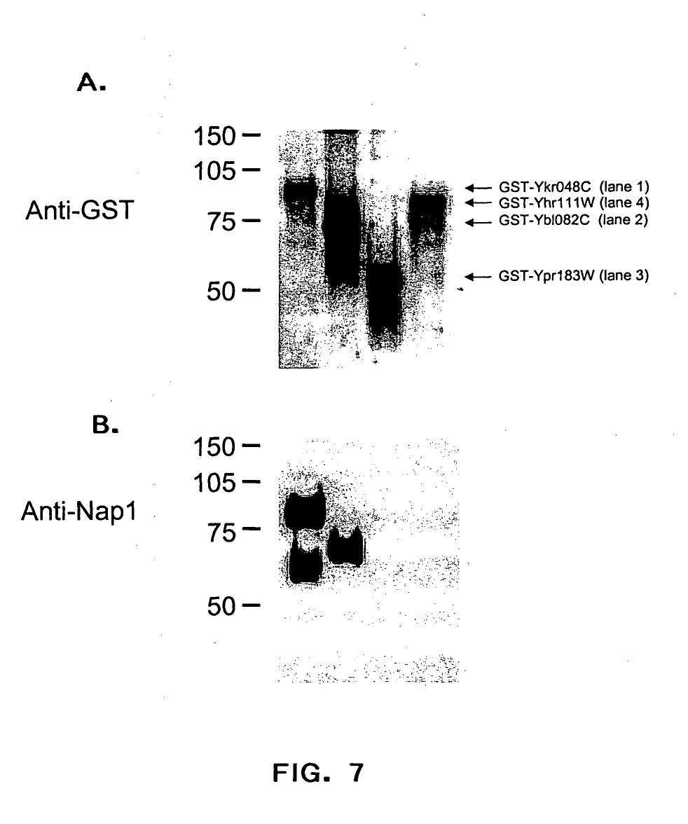

Anti-Nap1 was the least specific antibody examined, recognizing approximately 1770 proteins (FIGS. 1 and 6). Analysis of these signals indicated a direct correlation with the signals obtained with an anti-GST antibody for the same spots. Spots of pure GST on the array gave weaker signal intensities than may GST-fusion proteins present on the array in lower amounts, suggesting that anti-Nap1 is not binding specifically to the GST fusion tags. When several of the proteins that gave strong signals on the array were probed with anti-Nap1 on a Western blot, only some of the proteins were detected (FIG. 7), suggesting that the antibody may be recognizing a structured epitope on the proteins that could not be detected or the affinity/sensitivity of the antibody for these proteins is too low to detect via Western analysis.

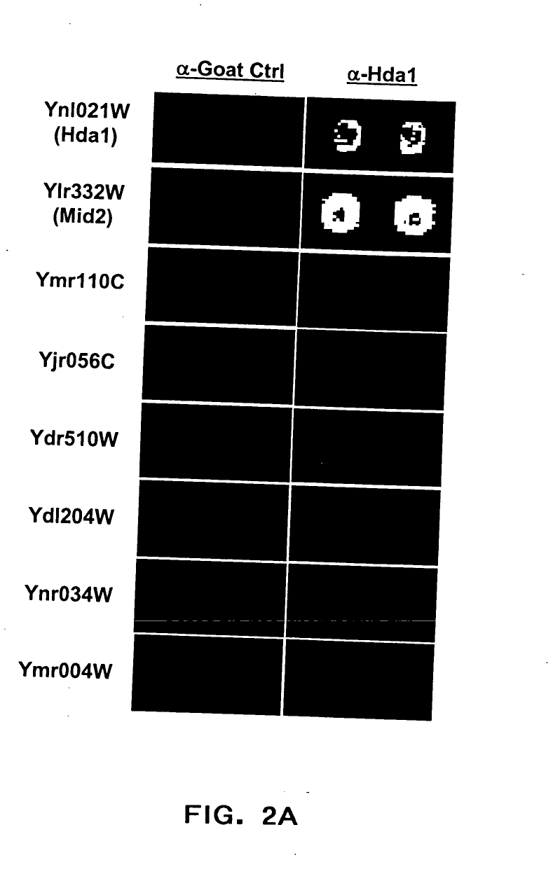

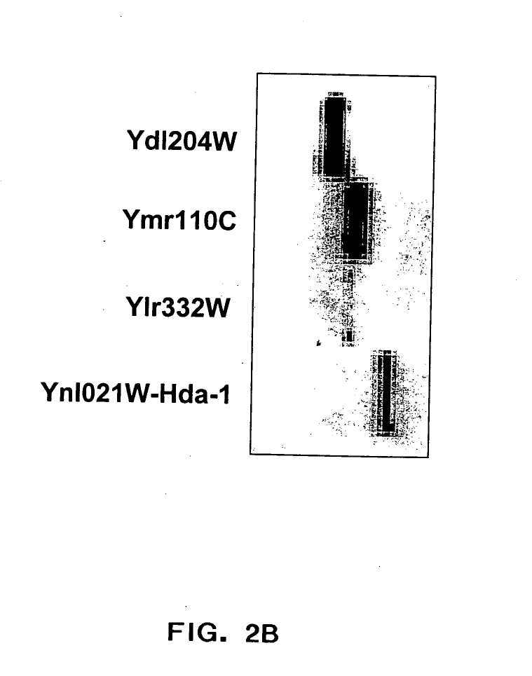

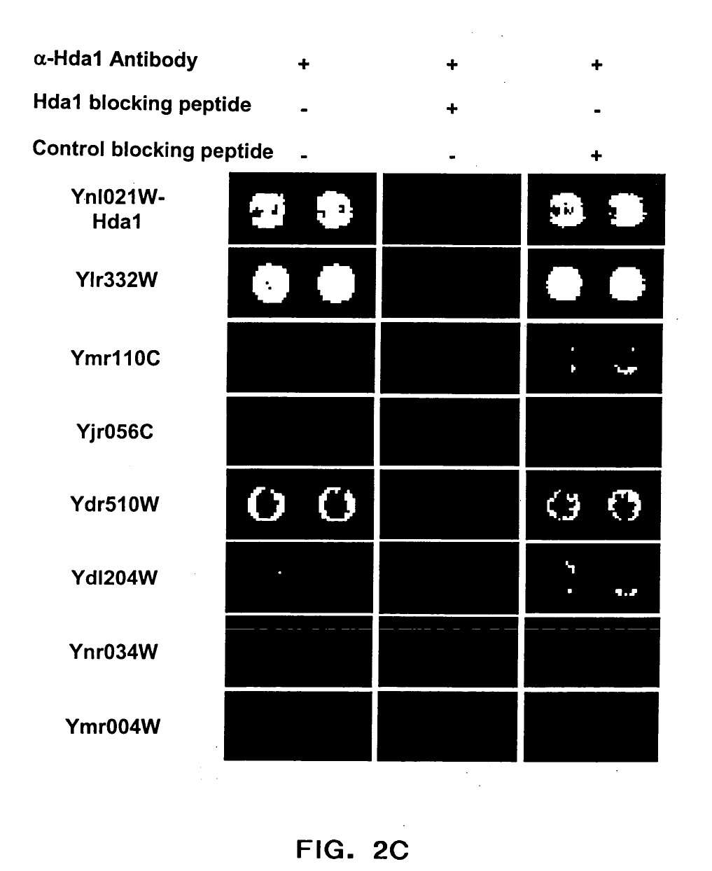

The anti-Hda1 antibody cross-reacts with seven different yeast proteins; FIG. 2A shows the signals obtained from the array spots representing these proteins. The cognate and non-cognate proteins were purified and separated on an SDS-PAGE gel, blotted to nitrocellulose and probed with the anti-Hda1 antibody. As shown in FIG. 2B, the anti-Hda1 antibody recognized its cognate protein Hda1 and three out of the seven other proteins observed to give significant signals on the arrays; thus, the anti-Hda1 antibody appears to recognize a common epitope(s) on the these proteins. The lack of signals by Western blotting of the other four proteins could be due to insufficient sensitivity or to the presence of a conformation-sensitive epitope that is disrupted in the denaturing gel.

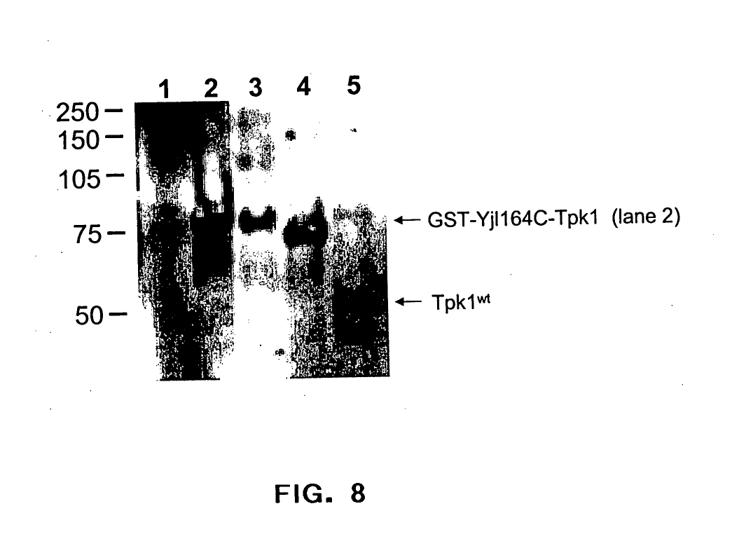

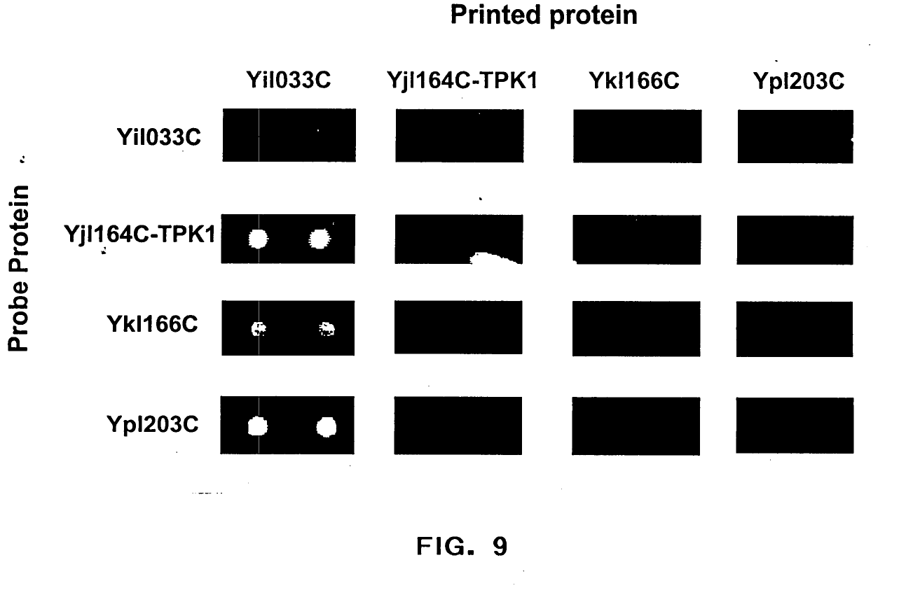

Tpk1 is a protein kinase involved in pseudohyphal growth and ras signal transduction (Robertson et al., 1998, Pro Natl Acad Sci USA 95, 13783-7; Tokiwa et al., 1994, Nature 371, 342-5). Significant signals were observed for eight proteins on the arrays probed with the antibody against this protein. Three of these proteins, Ykl166C-Tpk3 (a protein kinase), Ypl203W-Tpk2 (a protein kinase) and Yil033C-Bcy1 (a cAMP-dependent protein kinase inhibitor) have been shown by mass spectroscopy to be co-immunoprecipitated with Tpk1 (Ho et al., 2002, Nature 415, 180-3). Western blot analysis with the anti-Tpk1 antibody revealed a protein with the same molecular weight as Tpk1 co-purifying with Yil033C-Bcy1, but not with Ykl166C-Tpk3 or Ypl203W-Tpk2 (FIG. 8). This Western blot also showed, however, that the anti-Tpk1 antibody binds to the GST-fusions of these two proteins, suggesting that the signals observed for Ykl166C-Tpk3 and Ypl203W-Tpk2 on the array with the anti-Tpk1 antibody are due to cross-reactivity with a common epitope. These findings are in agreement with microarray-based protein interaction experiments that show the interaction between Tpk1 and Yil033C-Bcy1, but did not show interactions between Tpk1 and Ypl203W-Tpk2 or Ykl166C-Tpk3; Yil033C-Bcy1, however, also interacts with Ypl203W-Tpk2 and Ykl166C-Tpk3 on the array (FIG. 9).

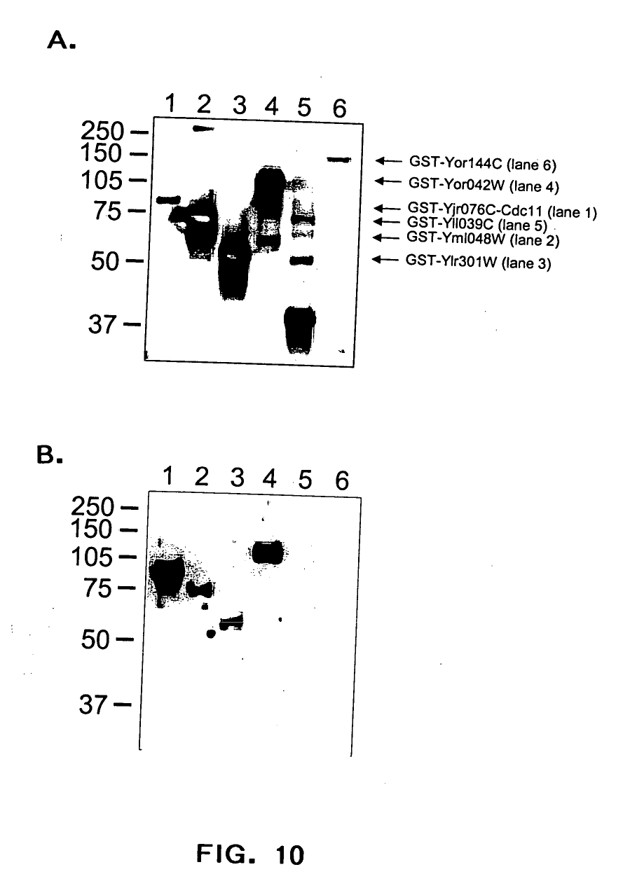

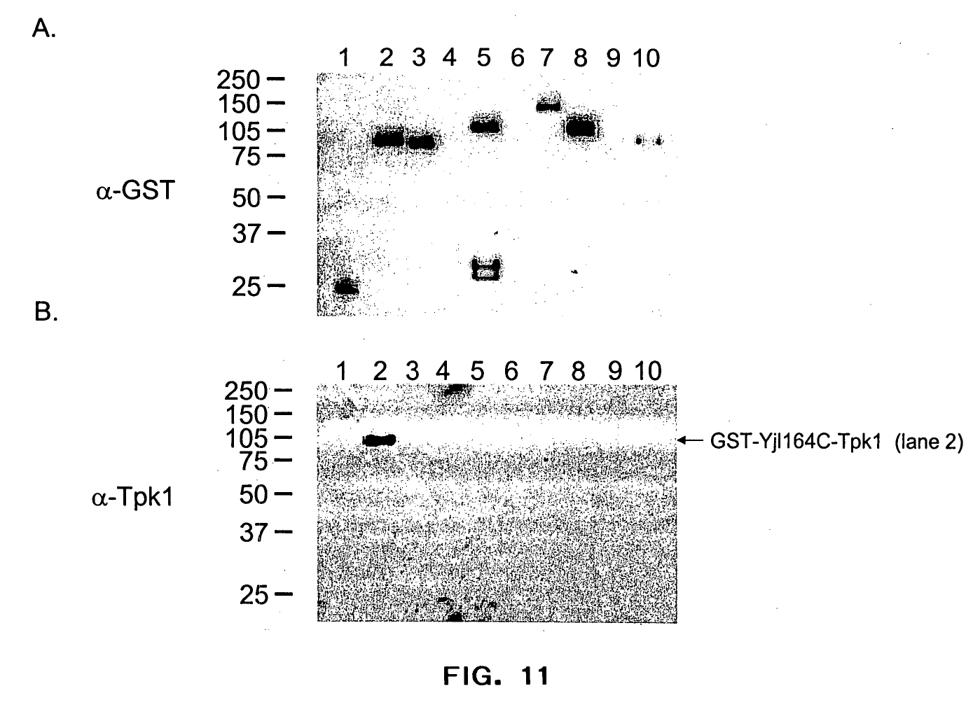

For anti-Cdc11 and anti-Hog1, six and one non-cognate proteins were observed on the yeast proteome array to have a signal-to-background ratio greater than 2.0, respectively. Three out of the six non-cognate proteins seen on the array probed with the anti-Cdc11 antibody could also be observed in a Western blot (FIG. 10). The anti-Hog1 antibody, however, was unable to detect on a Western blot the non-cognate protein observed on the array.

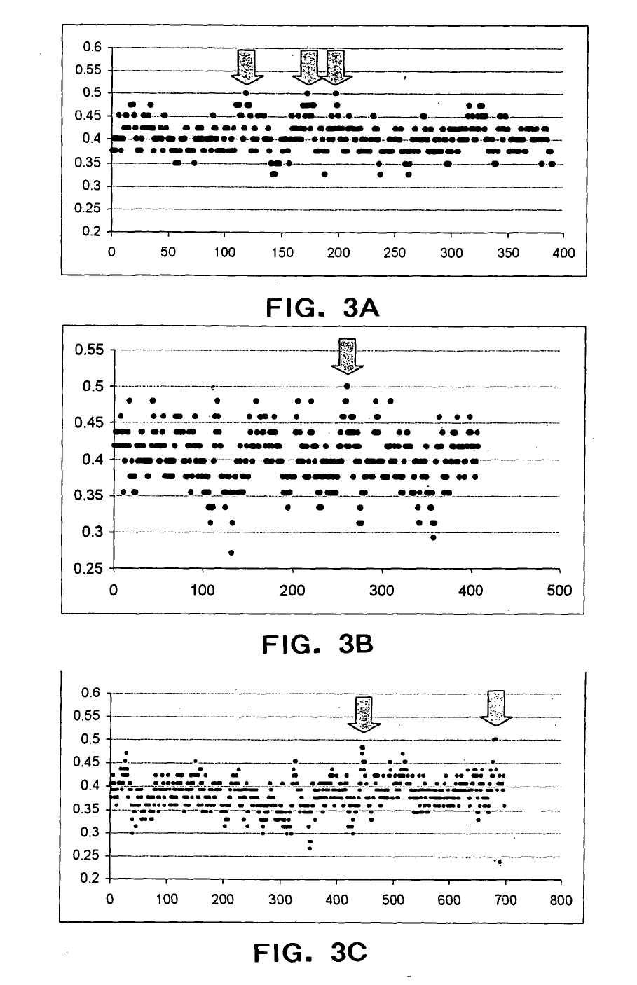

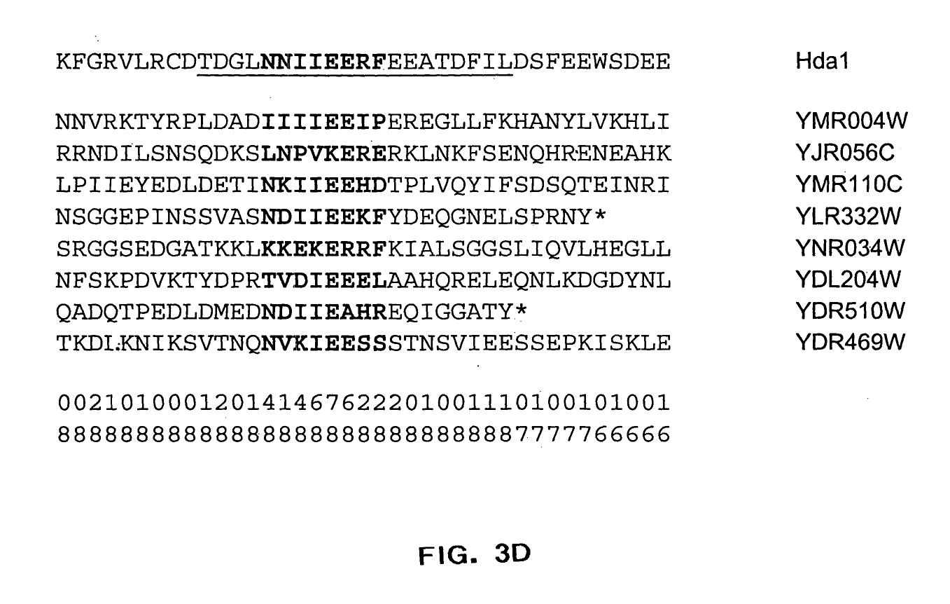

Because many of the antibodies described above were raised to linear epitopes (peptides), some degree of primary sequence similarity between the ‘target’ proteins and ‘cross-reactive’ proteins is expected. In order to test for this, a comprehensive search for short stretches of sequence homology was performed. In each case, an 8 amino acid window of the reference sequence (the ‘target’ protein) was scanned against all 8 amino acid stretches in each of the ‘cross reactive’ proteins. For each window, the best match for each cross-reacting protein was calculated, and the average identity was plotted as a function of reference sequence window (FIG. 3A-C). For each target protein, 1 to 3 regions of higher homology are apparent.

The analysis was followed up for the antibody targeted against Hda1, for which it was determined the antibody was raised against a 21 amino acid peptide with the sequence TDGLNNIIEERFEEATDFILD. Comparison of this sequence with the sequence cross comparison of the 7 reactive proteins shows that the region of highest similarity (see FIG. 3C) is entirely contained within the 21 amino acid sequence of the peptide (FIG. 3D). To confirm this peptide as a common epitope on the proteins that cross-react with the anti-Hda1 antibody, arrays were probed with the antibody in the presence of an excess amount of the immunizing peptide; a peptide of similar length but different sequence was used as a control. As shown in FIG. 2C, the Hda1 blocking peptide inhibits the interaction of anti-Hda1 with its cognate antigen Ynl021W-Hda1 as well to each of the seven cross-reacting proteins. No inhibition of anti-Hda1 binding to these proteins was observed with the control peptide.

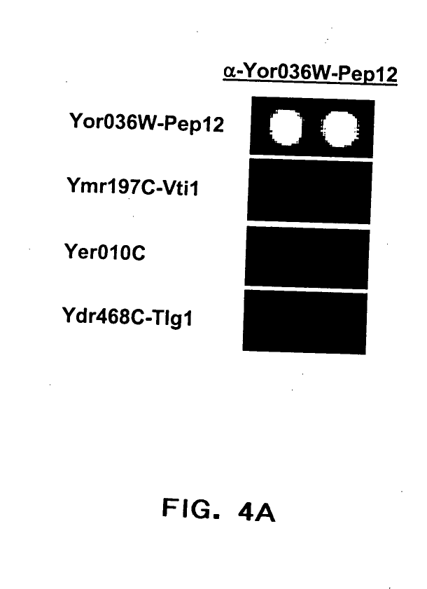

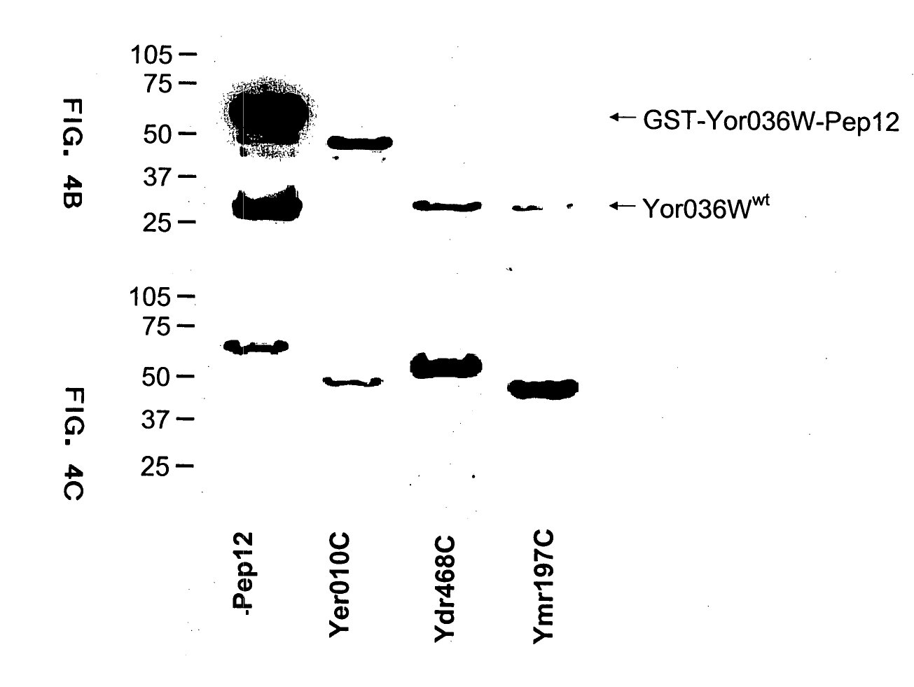

Analysis of monoclonal antibody cross-reactivity. Several monoclonal antibodies were also tested for specificity on yeast proteome microarrays. For both anti-Sed3 (FIG. 1) and anti-Cox4, only the GST fusions of the cognate proteins, GST-Cox4 and GST-Sed3, gave signals with their respective antibodies (Table 1). The anti-Pep12 antibody, however, exhibited binding with Ymr197C-Vti1, Yer010C and Ydr468C-Tlg1 in addition to the expected binding to Pep12 (FIG. 4A). Two-hybrid and affinity purification data indicate that Pep12 interacts with Ymr197C-Vti1 (von Mollard et al., 1997, J Cell Biol 137, 1511-24; Ito et al., 2001, Proc Natl Acad Sci USA 98, 4569-74), Ymr197C-Vti1 has been shown by co-immunoprecipitation to interact with Ydr468C-Tlg1 (Holthuis et al., 1998, EMBO J 17, 113-26). In contrast, no references in the literature could be found that supports an interaction between Pep12 and Yer010C. To test for the possibility that endogenous Pep12 co-purified with Ymr197C-Vti1 and Ydr468C-Tlg1, immunoblot analysis was carried out with all 4 proteins. In lane 1 of FIG. 4B and FIG. 4C, the 63 kDa GST-Pep12 fusion protein reacts with both anti-Pep12 and anti-GST antibodies, respectively. A smaller band of 33 kda, the expected molecular weight of endogenous Pep 12, is recognized by the anti-Pep12 antibody in lanes 1, 3 and 4 (FIG. 4B). The observation that anti-Pep12 and anti-GST both recognize a 55 kDa protein in lane 2 demonstrates that anti-Pep12 is cross-reacting with a common epitope on GST-Yer010C.

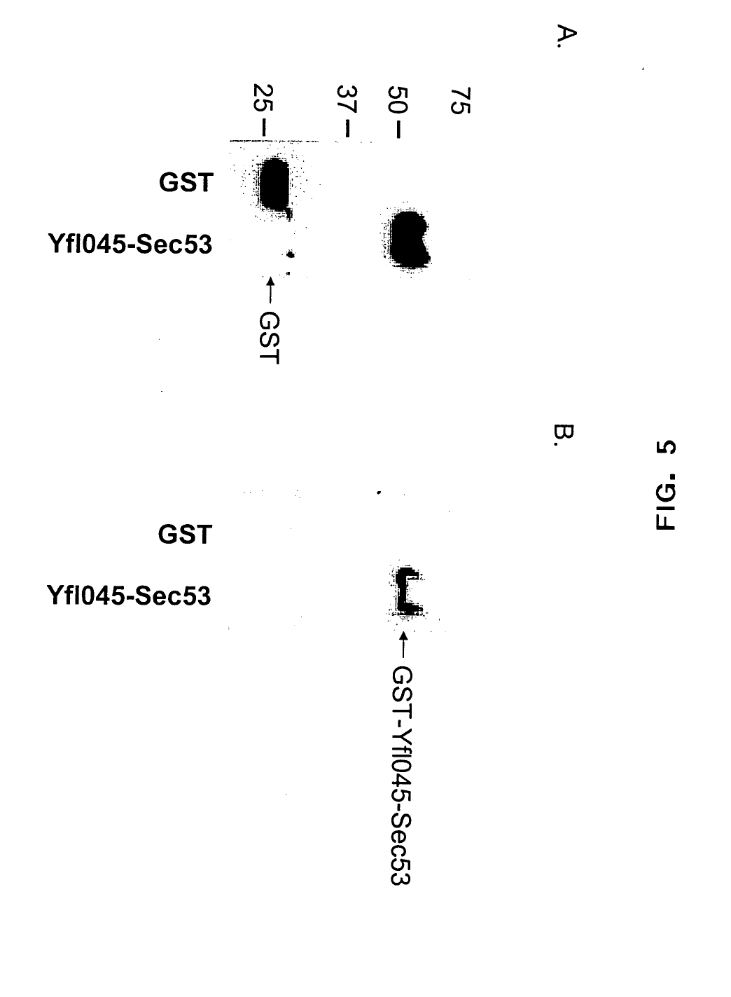

Antibodies against proteins not on the yeast proteome array. In addition to testing for cross-reactivity with antibodies directed against proteins present on the yeast proteome array, we also tested antibodies against proteins not present on the array. Monoclonal antibodies anti-HA (an antibody against the influenza hemagluttinin epitope), anti-MYC (an antibody against the Myc epitope), and anti-FLAG (an antibody against the synthetic FLAG epitope; Miceli et al., 1994, J Immunol Methods 167, 279-87) did not produce any significant signals for any protein on the array. Clb5 is a yeast protein that is present on the array, but did not give a detectable GST signal on the printed array. A polyclonal antibody against Clb5, however, detected a protein designated as Yfl045-Sec53. Western analysis of this protein shows that both anti-Clb and anti-GST detect a band at the predicted molecular weight of the GST fusion proteins (FIG. 5), demonstrating that Clb5 and Sec53 share a common epitope.

Discussion

In principal, a microarray containing most if not all of the proteins for a given organism is the ideal substrate for measuring the specificity of an antibody directed against a protein from that organism. In the case of the yeast proteome array utilized in this study, each protein is immobilized in defined locations on the array. Consequently, if an antibody cross-reacts with a protein on the array, the identity of the protein and its sequence are readily available. Furthermore, each protein is deposited in roughly equal amounts, thus affording a screening mechanism that is relatively unbiased in terms of the effect of protein concentration on sensitivity of cross-reactivity detection. Finally, whole proteome arrays allow the screening of large numbers of proteins in both their native and denatured forms since the arrays can be treated with various denaturants before probing with antibodies. All of these features give protein microarray-based screening of antibody specificity distinct advantages over more commonly used methods of screening. In immunoblot-based screening, for example, cross-reactive proteins have to be cut from gels and identified by sequencing or mass spectrometry (Yu et al., 2003, J Immunol 170, 445-53). Antibody screening using immunoblots of cell lysates or immunohistochemistry of tissue samples is also made more challenging by the much broader range of protein concentrations present, including many proteins that are only present in very low quantities. It was found that detection of proteins on microarrays is more sensitive than Western analysis, even when using the most sensitive Western blot detection kits.

The initial test of using proteome microarrays as an antibody screening tool was carried out using the yeast proteome microarray originally described by Zhu et al. (2001, Science 293:2101-2105) and antibodies directed against yeast proteins. In screening the yeast proteome array, it was found that the specificities of the antibodies tested ranged considerably. On one end of the specificity spectrum is the antibody directed against the protein Nap1, which appeared to recognize many hundreds of proteins on the array. The correlation of the signals obtained with anti-Nap1 and the amount of protein in the spot suggests the antibody or a component of the antibody preparation may recognize a common element in proteins such as a particular amino acid or a simple peptide sequence (e.g. di- or tri-peptide).

The majority of the anti-yeast protein antibodies examined in this study exhibited a degree of specificity that was in between that of the non-specific anti-Nap1 antibody and the four monospecific antibodies. A variety of publicly available multiple sequence alignment tools (e.g., T-Coffee (Notredame et al., 2000, J Mol Biol 302, 205-17) and PSI-BLAST, (George et al., 2002, Proteins 48, 672-81) were used in an attempt to define common epitopes; however, the ambiguity in these results demonstrated that a new algorithm for common epitope identification is required. This algorithm was designed to perform comprehensive screens for short defined regions of sequence similarity among a group of much larger sequences, thus enabling graphical visualization of all potential common epitopes, and simple identification of the best candidates. Using this novel approach, most of the cross-reactivity observed on the arrays can apparently be accounted for by the presence of common epitopes in the sequences of the proteins. For example, the sequence alignment algorithm was consistent with the Western analysis and peptide inhibition data in showing that certain proteins shared a common epitope with Hda1 (FIG. 2, FIG. 3). However, a sequence identity search against the yeast proteome for matches to the 21 amino acid immunizing peptide indicates that only 3 of the 7 cross-reactive proteins are among the top 1000 hits to the predicted yeast proteome of 6,300 proteins. Also, there are 86 yeast proteins which have better matches to the immunizing peptide than any of the cross-reactive proteins. Thus, the new sequence analysis algorithm is clearly useful for the identification of epitopes that confer cross-reactivity upon proteins. These findings indicate that a thorough empirical assessment of antibody cross-reactivity will be a necessary feature of any effort to produce large numbers of specific antibodies.

In contrast to the above examples, the cross-reactivity observed with the monoclonal antibody against Pep12 and the polyclonal antibody against Tpk1 cannot be accounted for strictly on the basis of common epitopes. Instead, Western analysis showed that some of the apparently cross-reactive protein preparations contained small but detectable amounts of the cognate protein. Previous reports have show the interactions between Pep12 and Vti1 and between Vti1 and Tlg1, but not between Pep12 and Tlg1, von Mollard et al. (1997, J Cell Biol 137, 1511-24); Ito et al. (2001, Proc Natl Acad Sci USA 98, 4569-74); and Holthuis et al. (1998, Embo J 17, 113-26). The demonstration of co-purification of Vti1 and Tlg1 with Pep12 suggest a model in which Tlg1 interacts indirectly with Pep12 through Vti1. Similarly, the co-purification of Tpk1 with Ykl166C and Ypl203W reported by Ho et al. (2002, Nature 415, 180-3) is most likely the result of indirect protein-protein interactions in with Tpk1 interacts with both Ypl203W-Tpk2 and Ykl166C-Tpk3 through Yil033C-Bcy1. This prediction is supported by the protein-protein microarray experiments which revealed an interaction of Tpk1 only with Yil033C-Bcy1 and by the observation that both Ypl203W-Tpk2 and Ykl166C-Tpk3 interact with only Yil033C-Byc1 on the arrays. This is the first time that interacting proteins have been purified and detected on microarrays.

The results presented here clearly demonstrate the utility of whole proteome microarrays for screening antibody specificity. The arrays plainly distinguished specific from non-specific antibodies; furthermore, the identification of the cross-reacting proteins was unambiguously established. This type of information should prove invaluable for correctly interpreting the results of the various kinds of biochemical analyses carried out using these antibodies. One unexpected finding was that antibody screening experiments of this type could be useful for revealing protein-protein interactions. It is expected that similar types of results would be obtained using arrays of proteins from different species. Proteome arrays will also be useful for evaluating the results of the many target validation studies that are carried out with antibodies in humans and other species. In addition, pre-screening anti-human antibodies on human proteome microarrays will become a critical part of the development of more specific and more effective antibodies for use in the clinic.

Material and Methods

Preparation of Yeast Protein Microarrays. Yeast proteins were purified as described in Zhu et al. (2001, Science 293:2101-2105). Proteins were immobilized on FAST (nitrocellulose pad size, 20mm x 60mm) slides by printing each protein in duplicate with a Genemachines Omnigrid arrayer. Each array contains 48 subarrays with 18×18 geometry with 250 μm center-to-center spacing. Cy-5-labeled rabbit antibody, glutathione-S-transferase (GST), bovine serum albumin (BSA) and biotinylated rabbit antibody, were included to facilitate data analysis.

Antibodies and Probing of Yeast Protein Microarrays. Antibodies against yeast proteins Hda1, Hog1, Tpk1, Mad2, Cdc42, Clb5, Cdc11 and Nap1 were obtained from Santa Cruz Biotechnology, Inc. (Santa Cruz, Calif.). Antibodies against yeast proteins Sed3, Cox4 and Pep12 were obtained from Molecular Probes (Eugene, Oreg.). Antibodies against HA and Myc were obtained from Covance, Inc (Princeton, N.J.), and the antibody against FLAG was obtained from Sigma (St. Louis, Mo.). Slides were blocked with 1% BSA in TBST and subsequently probed with antibodies in PBS, 5 mM MgCl₂, 0.05% Triton X-100, 5% glycerol, 1% BSA for 2hrs. After 3 washes, secondary antibodies (anti-goat, anti-rabbit or anti-mouse conjugated to CyS (Jackson Immunoresearch Laboratory, West Grove, Pa.) were added and incubated for 1 hour. After three washes, slides were dried and images acquired with an Axon 4000B scanner at a PMT setting of 600. Images were analyzed with GenePix 4.0 and data processed with Microsoft Excel.

SDS-PAGE and Western analyses of Protein Samples. Purified proteins were mixed with sample buffer, heated and run in 5%/10% SDS-PAGE gels, transferred to nitrocellulose and blocked overnight with 1% BSA in TBST. Subsequently, blots were probed with primary antibodies for 2 hours, washed three times with TBST, probed with secondary antibodies conjugated to HRP for 1 hour and washed three times with TBST. Femto-reagent (Pierce Co., Milwaukee) was added and images acquired with an Alpha innotech Imaging station.

Protein Sequence Analysis. Protein sequences were analyzed using custom software which comprehensively compares all n-amino acid windows of sequence homology between a reference sequence (the ‘target’ protein) and all n-amino acid stretches in a defined set of ‘cross-reactive’ proteins. At each sequential window, the highest identity hit for each cross-reactive protein was recorded and averaged for all cross-reactive proteins. Unlike most sequence alignment strategies, this approach represents a systematic comprehensive search for alignments of short sequences of defined length between much longer sequences, and is therefore particularly useful in identifying shared epitopes.

Amino Acid Compostion of Hda1 blocking peptide. Anti-Hda1 blocking peptide was purchased from Santa Cruz Biotechnologies, Inc (Santa Cruz, Calif.; 0.2 ug/ul in 1XPBS). Peptide was analyzed for amino acid composition using a Beckman 7300 amino acid analyzer at the HHMI Biopolymer Keck Foundation Bioresearch Laboratory at Yale University. Comparison of the amino acid composition with the linear sequence of Hda1 was used to determine the peptide sequence: TDGLNNIIEERFEEATDFILD.

TABLE 1
Antibodies Used for Probing Yeast Protoarray

| | ˜Amount of | Source of | | | Ab Probing | | Number of Proteins with |
|---|---|---|---|---|---|---|---|
| Antibody | Protein (pg) | Epitope(s) | Nature of Immunogen | Antibody | Concentration | | Signal/Noise >2.0 |
| Ynl021W-Hda1 | 0.3 | yeast | peptide20a.a.'s | polyclonal† | 0.8 | ug/ml* | 8 |
| Ylr113W-Hog1 | 0.4 | yeast | peptide20a.a.'s | polyclonal† | 0.8 | ug/ml | 1 |
| Yjl164C-Tpk1 | 1.2 | yeast | peptide20a.a.'s | polyclonal† | 0.8 | ug/ml* | 9 |
| Yjl030W-Mad2 | 2.3 | yeast | peptide20a.a.'s | polyclonal† | 0.8 | ug/ml* | 1 |
| Ylr229C-Cdc42 | 5.2 | yeast | peptide20a.a.'s | polyclonal† | 0.1 | ug/ml* | 1 |
| Ypr120C-Clb5 | Not detectable | yeast | peptide20a.a.'s | polyclonal† | 0.1 | ug/ml | 1 |
| Yjr076C-Cdc11 | 2.2 | yeast | protein1-415a.a.'s | polyclonal‡ | 0.04 | ug/ml* | 7 |
| Ykr048C-Nap1 | 7.2 | yeast | protein1-417a.a.'s | polyclonal‡ | 0.02 | ug/ml* | 1770 |
| Ypr183W-Sed3 | 13.3 | yeast | proteincytsolicdomain | monoclonal£ | 2.0 | ug/ml* | 1 |
| Ygl187C-Cox4 | 0.9 | yeast | protein | monoclonal£ | 2.0 | ug/ml* | 1 |
| Yor036W-Pep12 | 4.4 | yeast | Protein C-terminus | monoclonal£ | 2.0 | ug/ml* | 4 |
| HA | — | Influenza | peptide12a.a.'s | monoclonal£ | 1 | ug/ml | 0 |
| MYC | — | human | protein | monoclonal£ | 1 | ug/ml | 0 |
| FLAG | — | nonyeast | peptide | monoclonal£ | 2.4 | ug/ml | 0 |

*Ab probing concentration was titered such that reactivity of antibody with cognate protein was at or near scanner saturation
†goat IgG isotype
‡rabbit IgG isotype
£mouse IgG isotype

All antibodies were purchased from commercial vendors (See experimental protocols). For each antibody, the amount of cognate antigen present on the array by [PUT DESCRIPTION OF HOW GST WAS USED TO CALCULATE AMOUNT OF PROTEIN IN METHODS AND REFER TO IT HERE]. The antibody concentration used to probe the protein arrays was determined by titering each antibody for maximal reactivity with its cognate antigen. The number of proteins having a signal to background ratio greater than or equal to 2.0 is reported.

### Example 2

### Epitope Searching

Yeast ProtoArray experiments have demonstrated significant cross reactivity of a polyclonal antibody directed against HDA1 (YNL021W) with a number of other proteins (YDR469W, YDL204W, YMR110C, YLR332W). A ‘naive’ search for short stretches of sequence homology among these proteins was performed in an attempt to identify a common epitope.

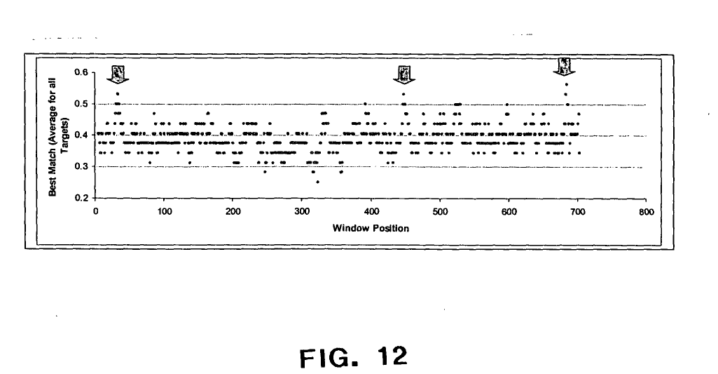

8 amino acid windows of the ‘reference’ sequence, YNLO2 1W, were scanned against all 8 amino acid stretches in each of the ‘cross-reactive’ sequences (YDR469W, YDL204W, YMR110C, YLR332W). For each window, the best match with each cross-reactive sequence was calculated, and the average identity was plotted as a function of reference sequence window (FIG. 12). From this analysis, 3 regions of highest homology are identified (arrows). An alignment of these sequences is presented in Table 2. The 8 amino acid window from the best matching region (region 3) is fully contained within a 20 (21?) amino acid peptide which blocks the interaction of the antibody with all of these proteins (Table 3). Thus, comparative sequence analysis has utility in identifying and explaining the mechanism of cross-reactivity.

In order to assess the utility of sequence analysis in predicting cross reactivity, all yeast proteins were searched for either the 8 amino acid epitope core sequence NNIIEERF or the 20/21 amino acid immunogenic peptide sequence TDGLNNIIEERFEEATDFILD. The top matches are presented in Table 4. In addition to the observed cross-reactive proteins, a large number of proteins are identified with similarly high sequence conservation which show no empirical evidence of cross reactivity. Thus, although sequence analysis is useful in explaining the observed cross-reactivity, it is clearly insufficient to predict it.

TABLE 2

Sequence alignments for the 3 regions of
highest homology based on a comprehensive 8
amino acid window sequence comparison. Sequence
alignment is shown for 12 amino acids - the 8
amino acid core (bold in all, underlined for
reference sequence YNL021W) and 2 amino acids
on both N′ and C′. Identities are in
red.

| | | |
|---|---|---|
| Sequence | Protein | Identity (in 8 aa core) |

| | | |
|---|---|---|
| Region 1 | | |
| EEENSLSTTSKS | YNL021W | |
| ESEESSSTNSVI | YDR469W | .625 |
| EQADSSSLTSFS | YLR332W | .5 |
| VMENLLTTAGVS | YMR110C | .5 |
| TDEGSYSTSIKS | YDL204W | .5 |
| Region 2 | | |
| FNEPINDSIISK | YNL021W | |
| GGEPINSSVASN | YLR332W | .625 |
| KNEPYIDKIISK | YDL204W | .625 |
| FNETINKIIESK | YMR110C | .5 |
| MNYLIEQSNILK | YDR469W | .375 |
| Region 3 | | |
| GLNNIIEERFEE | YNL021W | |
| ASNDIIEEKFYD | YLR332W | .75 |
| TINKIIEEHDTP | YMR110C | .625 |
| NQNVKIEESSEP | YDR469W | .5 |
| NLFNNRHENFDE | YDL204W | .375 |

TABLE 3

Sequence alignment of the immunogenic peptide
region with best matches from each of the 4
‘cross-reactive’ proteins. The 8 amino
acid core from region 3 (FIG. 1) is in bold for
all sequences, and underlined in the reference
sequence

| Sequence | Protein |
|---|---|
| TDGLNNIIEERFEEATDFILD | YNL021W |
| SVASNDIIEEKFYDEQGNELS | YLR332W |
| KDFHRNKIESVLNETTKLMND | YMR110C |
| FHKNYNKVVEKTEPYIDKIIP | YDL204W |
| SSSTNSVIEESSEPKISKLEN | YDR469W |

TABLE 4

| | | |
|---|---|---|
| Sequence | Protein | Identity |

| | | |
|---|---|---|
| TDGLNNIIEERFEEATDFILD | YNL021W | 1.000 |
| TNGRNIIIEEIEASRTSFTLY | YDR291W | 0.476 |
| TDYLKNIIVENSGTSGDEDVD | YIL075C | 0.429 |
| RDYLNSYIEERLQEEHLDINN | YKL201C | 0.429 |
| KTDLVNFIEERFKTFCDEELE | YKR054C | 0.429 |
| TVLENKKIEEGKETAVDREED | YKL188C | 0.429 |
| IEGLNIISSGTFESLQDFVLQ | YNL193W | 0.429 |
| TDASNGYDEELPEEEQEFSDD | YNL124W | 0.429 |
| SYYLNCIIEENFKEMTRKLQR | YNL126W | 0.429 |
| GQFLENFLELNLNEVTDLIRD | YDR481C | 0.381 |
| TLSAGNACPGWDEDANDDILD | YBR092C | 0.381 |
| TDIFKNCLENQFEITNLKILF | YKL057C | 0.381 |
| DDDDDDEDEEEEEEVTDQLED | YFR033C | 0.381 |
| VDGKGNETEEDDIKFIKGILD | YJL168C | 0.381 |
| DDGLPNGITLIGKKFTDYALL | YBR208C | 0.381 |
| TISLIHEIEKIFEEDIHFEQN | YHR184W | 0.381 |
| FQGGLDIIKESLEEDPDFLQH | YDR098C | 0.381 |
| TDYLFDYREVLESLGLDIILD | YLR443W | 0.381 |
| QFLLSKIIEARISGAFFEIWD | YDL231C | 0.381 |
| TEFYNNYSMQVREDERDYILD | YDL040 | 0.381 |

### 7. References Cited

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A method for predicting at least part of a binding site in a target protein, wherein said binding site can be bound by a molecule, said method comprising:
(a) comparing, for each of a plurality of cross-reactive proteins, each of a first plurality of amino acid sequences in a region of said target protein with each of a second plurality of amino acid sequences in a region of said cross-reactive protein, wherein each said cross-reactive protein can be bound by said molecule;
(b) identifying an amino acid sequence in said first plurality of amino acid sequences that exhibits the highest average sequence homology score, said average score being based upon the sequence homologies to an amino acid sequence in each of said second plurality of amino acid sequences in regions of said cross-reactive proteins, wherein said identified amino acid sequence in said first plurality of amino acid sequences is predicted to be said at least part of a binding site in said target protein.

2. A method for predicting at least part of a binding site in a target protein, wherein said binding site can be bound by a molecule, said method comprising:
(a) evaluating the degree of homology between each n-amino acid window of a plurality of n-amino acid windows of the target protein with each n-amino acid window of a plurality of n-amino acid windows of a first cross-reactive protein of a plurality of cross-reactive proteins, wherein (i) each cross-reactive protein in the plurality of cross-reactive proteins can be bound by the molecule, and (ii) n is between 6 and 25;
(b) performing step (a) for each cross-reactive protein in the plurality of cross-reactive proteins;
(c) identifying, for each n-amino acid window in the target protein, the highest degree of sequence homology with an n-amino acid window in a cross-reactive protein for each cross-reactive protein;
(d) identifying the n-amino acid window(s) in the target protein that have the highest average of the highest degrees of sequence homologies identified in step (c), wherein said identified n-amino acid window(s) comprises at least part of the binding site(s) in the target protein.

3. A method for predicting at least part of a binding site in a target protein, wherein said binding site can be bound by a molecule, said method comprising:
(a) comparing each n-amino acid window in a plurality of n-amino acid windows of the target protein with each n-amino acid window in a plurality of n-amino acid windows of a first cross-reactive protein of a plurality of cross-reactive proteins, wherein (i) each cross-reactive protein in the plurality of cross-reactive proteins can be bound by the molecule, and (ii) n is between 6 and 25;
(b) assigning a score for each n-amino acid window comparison of step (a), wherein the score reflects the degree of sequence homology between the two n-amino acid windows compared;
(c) performing steps (a) and (b) for each cross-reactive protein in the plurality of cross-reactive proteins;
(d) identifying the highest scores assigned in step (b) of each n-amino acid window in the target protein for each cross-reactive protein; and
(e) identifying the n-amino acid window(s) in the target protein that have the highest average score(s), wherein said identified n-amino acid window(s) comprises at least part of the binding site(s) in the target protein.

4. The method of claim 1, 2, or 3, wherein the binding site is an epitope and the molecule is an antibody.

5. The method of claim 1, 2 or 3, wherein the degree of sequence homology reflects the degree of sequence identity.

6. The method of claim 1, 2 or 3, wherein the degree of sequence homology reflects the degree of sequence similarity.

7. The method of claim 1, wherein the first plurality of amino acid sequences comprises successive overlapping amino acid sequences spanning said region of said target protein.

8. The method of claim 1, wherein said plurality of amino acid sequences of each said cross-reactive protein comprises successive overlapping amino acid sequences spanning said region of said cross-reactive protein.

9. The method of claim 7, wherein said successive overlapping amino acid sequences pan said region of said target protein at an amino acid interval of 1 amino acid.

10. The method of claim 8, wherein said successive overlapping amino acid sequences span said region of said cross-reacting protein at a amino acid interval of 1 amino acid.

11. The method of claim 2 or 3, wherein the plurality of n-amino acid windows in the target protein comprises successive, overlapping amino acid sequences spanning a region of the target protein.

12. The method of claim 2 or 3, wherein the plurality of n-amino acid windows in each cross-reactive protein comprises successive overlapping amino acid sequences spanning a region of the cross-reactive protein.

13. The method of claim 11, wherein said successive overlapping amino acid sequences span said region of said target protein at an amino acid interval of 1 amino acid.

14. The method of claim 12, wherein said successive overlapping amino acid sequence span said region of said cross-reactive protein at an amino acid interval of 1 amino acid.

15. The method of claim 1 or 11, wherein the region of the target protein has been identified as containing the binding site.

16. The method of claim 1 or 12, wherein the region of the cross-reactive protein has been identified as containing the binding site.

17. The method of claim 1 or 11, wherein the region of the target protein consists of the entire contiguous amino acid sequence of the target protein.

18. The method of claim 1 or 12, wherein the region of the cross-reactive protein consists of the entire contiguous amino acid sequence of the cross-reactive protein.

19. The method of claim 1 or 11, wherein the region of the target protein has been identified as being on the surface of the folded target protein.

20. The method of claim 1 or 12, wherein the region of the cross-reactive protein has been identified as being on the surface of the folded cross-reactive protein.

21. The method of claim 1, 2, or 3, wherein the method is computer-implemented.

22. A computer system comprising a processor, and a memory coupled to said processor and encoding one or more programs, wherein said one or more programs cause the processor to carry out the method of any one of claims 1, 2, and 3.

23. A computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method of any one of claims 1, 2, and 3.


SEQUENCE LISTING (50 sequences)

SEQ ID NO: 1 - protein, 8 aa, Yeast
NNIIEERF

SEQ ID NO: 2 - protein, 12 aa, Yeast
EEENSLSTTSKS

SEQ ID NO: 3 - protein, 12 aa, Yeast
ESEESSSTNSVI

SEQ ID NO: 4 - protein, 12 aa, Yeast
EQADSSSLTSFS

SEQ ID NO: 5 - protein, 12 aa, Yeast
VMENLLTTAGVS

SEQ ID NO: 6 - protein, 12 aa, Yeast
TDEGSYSTSIKS

SEQ ID NO: 7 - protein, 12 aa, Yeast
FNEPINDSIISK

SEQ ID NO: 8 - protein, 12 aa, Yeast
GGEPINSSVASN

SEQ ID NO: 9 - protein, 12 aa, Yeast
KNEPYIDKIISK

SEQ ID NO: 10 - protein, 12 aa, Yeast
FNETINKIIESK

SEQ ID NO: 11 - protein, 12 aa, Yeast
MNYLIEQSNILK

SEQ ID NO: 12 - protein, 12 aa, Yeast
GLNNIIEERFEE

SEQ ID NO: 13 - protein, 12 aa, Yeast
ASNDIIEEKFYD

SEQ ID NO: 14 - protein, 12 aa, Yeast
TINKIIEEHDTP

SEQ ID NO: 15 - protein, 12 aa, Yeast
NQNVKIEESSEP

SEQ ID NO: 16 - protein, 12 aa, Yeast
NLFNNRHENFDE

SEQ ID NO: 17 - protein, 21 aa, Yeast
TDGLNNIIEERFEEATDFILD

SEQ ID NO: 18 - protein, 21 aa, Yeast
SVASNDIIEEKFYDEQGNELS

SEQ ID NO: 19 - protein, 21 aa, Yeast
KDFHRNKIESVLNETTKLMND

SEQ ID NO: 20 - protein, 21 aa, Yeast
FHKNYNKVVEKTEPYIDKIIP

SEQ ID NO: 21 - protein, 21 aa, Yeast
SSSTNSVIEESSEPKISKLEN

SEQ ID NO: 22 - protein, 21 aa, Yeast
TDGLNNIIEERFEEATDFILD

SEQ ID NO: 23 - protein, 21 aa, Yeast
TNGRNIIIEEIEASRTSFTLY

SEQ ID NO: 24 - protein, 21 aa, Yeast
TDYLKNIIVENSGTSGDEDVD

SEQ ID NO: 25 - protein, 21 aa, Yeast
RDYLNSYIEERLQEEHLDINN

SEQ ID NO: 26 - protein, 21 aa, Yeast
KTDLVNFIEERFKTFCDEELE

SEQ ID NO: 27 - protein, 21 aa, Yeast
TVLENKKIEEGKETAVDREED

SEQ ID NO: 28 - protein, 21 aa, Yeast
IEGLNIISSGTFESLQDFVLQ

SEQ ID NO: 29 - protein, 21 aa, Yeast
TDASNGYDEELPEEEQEFSDD

SEQ ID NO: 30 - protein, 21 aa, Yeast
SYYLNCIIEENFKEMTRKLQR

SEQ ID NO: 31 - protein, 21 aa, Yeast
GQFLENFLELNLNEVTDLIRD

SEQ ID NO: 32 - protein, 21 aa, Yeast
TLSAGNACPGWDEDANDDILD

SEQ ID NO: 33 - protein, 21 aa, Yeast
TDIFKNCLENQFEITNLKILF

SEQ ID NO: 34 - protein, 21 aa, Yeast
DDDDDDEDEEEEEEVTDQLED

SEQ ID NO: 35 - protein, 21 aa, Yeast
VDGKGNETEEDDIKFIKGILD

SEQ ID NO: 36 - protein, 21 aa, Yeast
DDGLPNGITLIGKKFTDYALL

SEQ ID NO: 37 - protein, 21 aa, Yeast
TISLIHEIEKIFEEDIHFEQN

SEQ ID NO: 38 - protein, 21 aa, Yeast
FQGGLDIIKESLEEDPDFLQH

SEQ ID NO: 39 - protein, 21 aa, Yeast
TDYLFDYREVLESLGLDIILD

SEQ ID NO: 40 - protein, 21 aa, Yeast
QFLLSKIIEARISGAFFEIWD

SEQ ID NO: 41 - protein, 21 aa, Yeast
TEFYNNYSMQVREDERDYILD

SEQ ID NO: 42 - protein, 39 aa, Yeast
KFGRVLRCDTDGLNNIIEERFEEATDFILDSFEEWSDEE

SEQ ID NO: 43 - protein, 39 aa, Yeast
NNVRKTYRPLDADIIIIEEIPEREGLLFKHANYLVKHLI

SEQ ID NO: 44 - protein, 39 aa, Yeast
RRNDILSNSQDKSLNPVKERERKLNKFSENQHRENEAHK

SEQ ID NO: 45 - protein, 39 aa, Yeast
LPIIEYEDLDETINKIIEEHDTPLVQYIFSDSQTEINRI

SEQ ID NO: 46 - protein, 34 aa, Yeast
NSGGEPINSSVASNDIIEEKFYDEQGNELSPRNY

SEQ ID NO: 47 - protein, 39 aa, Yeast
SRGGSEDGATKKLKKEKERRFKIALSGGSLIQVLHEGLL

SEQ ID NO: 48 - protein, 39 aa, Yeast
NFSKPDVKTYDPRTVDIEEELAAHQRELEQNLKDGDYNL

SEQ ID NO: 49 - protein, 29 aa, Yeast
QADQTPEDLDMEDNDIIEAHREQIGGATY

SEQ ID NO: 50 - protein, 39 aa, Yeast
TKDLKNIKSVTNQNVKIEESSSTNSVIEESSEPKISKLE